SEQUENCE LISTING (35 sequences)

SEQ ID NO: 1 - DNA, 2939 bp, Artificial Sequence
  PLE-wu transposon sequence
ttaacccttttgcagttagagggaatatttttcccaccaataaaaaccaataaaaaaggaacaaaatttctatttgcaattttatttttcgacctgacaacattaacgtaacattatgacatttgtttctgattttatttgacattgacagattcaattttttttcaaagtcttgaggtgaatttgaaaactagcggttttttgtacttcgagagaaatcggtacatatctttgcaatagagtggtgaaaaaaggtaagttcttctagatgttgtgcttttgaatgttttttcatagatataagacgtattttttatgtatctgccacgaattttcatatatgtcgattttttttggtgggaagaatattccctctaaccgtgaagtatccgctttctactaatacgtaaggttagagaaaatattcctttattatgtttacggtacctgtttttattgcttgcggctttttctcggggcttggtgtggtttttgcatggcttttgtttttttatttgcagattgagtgaaaatgaaaatacttagagatgacgatattgagaaggagttgaatggtatgttcggtttgtcagaaccggaagagtctgaagacggcgaagaatcagaagacgatgacgttccgtacaatgtcactaagttacagcgaattttaggggatttagaagaaccagaccttcgatctactgcaacaattcgagcatcgttatcgcctcaactatcaacatcatctgatgcggagatcccatgcggtgagcctttacttgtaatacctccacctactgtgatagaagctgttgcgtctacgtctgcagtgccattacatgtgccgcaaccagctgtactactgcctgctgagcctcctatttcacgtaagttgcctgtaaaaggtagaggacgaggagctaggatggaacgtggatccggaacgggacgtagatttagaacaggacgagaagctggaactagaatgggacgtggagctagaacgaggctaggaagtggggttggagataaagacagcgattcatccgaagataatacgccaccagtatcagaagcagagtcagaagcaagcgattctgacagagatgaagaagagtggaaaaaaaccttgtggactgatattaggccacagcttgagtcctttgatagtgttccaatgactccgacacgcgtgttaccatctaatgccaggcctatccgatattttgaaaaattttttactcaagaagtttttgaattaattatcacagagactaacagatatgcatgtcaaaacaacgtaataggctggaccatactagacataaaggaattgaaggctttcctaggtatattgattattatgggatacaatattttacctactttcgaattgtattggtcatcggatcccgaatttagagttgacgaaatagcttccaccatgacattccgaagattcaagcaaattttacgttgtttgcatctaaatgataattctaaacaacctgccagacttagccccgagtatgataagctattcaaaattcgcccgttattgacgcttataaacacctcttttcaagaaaatgcgcacaactcttcctctcagtcaatagatgaatcaatgattcttttcaaaggaaggtctacattgaagcaatacatgcctatgaagccaataaaaaggggtttcaaggtatggtgccgctgtgatagtataactggttatctgtacgaatttgatatatacaccggaagagatggtgacagagtagaagacaatttaggaggaaaagtggtcaaaaagttaacagaaaaattgaaaggaatggcggcagtgcatgttacatttgataattttttttgcagttatgatatcatgaattatctacatgtaaatggtatttctgcgagcggtactgtacgcaggcaaagagcagacttaccgaaacttgtgaaaagtaaaaaaaaactaaaactaacaaaagggcagtacaaatggagagtgaaggagaacgtggcttttgtaatctggcaagatacaaaagaagtcttatttatgaccaatgctttccatcccaaggacaatgaaacatctctacccaggaaaggaagagacggttccaagacagatgtaaggtgtcctgcagtagtgaaagaatatacaaaaagaatgggtggagtagatcactttgatcacatcaaagggacatactcagtggggcgaaggagcaaacggtggtggcttcgcatattttattttatttttgatgcctgcatcacaaattcttttttactccagggaaagaatgcgaatgcaaccaaattgtccaacctagagtatcgtgtcgctctagccagaggacttattggttgtttttcttcccgaaaacgtcgtgctgaaggcgtcaattacgtcgtaagaaaaaaagttgctgtatctgaaaattatcaaaaggcaattcacatcgttgcaccggaaattcgcttttccaacgttggtgatcacatgccaaatgatataccgtcttatcaaagatgccgatattgcagcactaaggcaaaagataagcgatctaagatcaagtgtagcaaatgtggagtaccgttatgtataacaccatgcttttcgaattttcacaaacaagtttaattttcttttatttttgtttctattttcttattggtattcaattgtgtgattgaaataaagttattatgtgagatttatgttgtttttttagtttgttgaaaaatcatactacaatttcattactctcaaacaggtagagggaatattcttaccaccaattaaaaaaaaaagtttaaaaaaaattgaatctcaatttttattttaggcatcttatgtaacacaaatccgaaaaccaaatctttctatctcatctagatccttataaccgttaaagggttaa

SEQ ID NO: 2 - protein, 708 aa, Artificial sequence
  PLE-wu transposase amino acid sequence
MKILRDDDIEKELNGMFGLSEPEESEDGEESEDDDVPYNVTKLQRILGDLEEPDLRSTATIRASLSPQLSTSSDAEIPCGEPLLVIPPPTVIEAVASTSAVPLHVPQPAVLLPAEPPISRKLPVKGRGRGARMERGSGTGRRFRTGREAGTRMGRGARTRLGSGVGDKDSDSSEDNTPPVSEAESEASDSDRDEEEWKKTLWTDIRPQLESFDSVPMTPTRVLPSNARPIRYFEKFFTQEVFELIITETNRYACQNNVIGWTILDIKELKAFLGILIIMGYNILPTFELYWSSDPEFRVDEIASTMTFRRFKQILRCLHLNDNSKQPARLSPEYDKLFKIRPLLTLINTSFQENAHNSSSQSIDESMILFKGRSTLKQYMPMKPIKRGFKVWCRCDSITGYLYEFDIYTGRDGDRVEDNLGGKVVKKLTEKLKGMAAVHVTFDNFFCSYDIMNYLHVNGISASGTVRRQRADLPKLVKSKKKLKLTKGQYKWRVKENVAFVIWQDTKEVLFMTNAFHPKDNETSLPRKGRDGSKTDVRCPAVVKEYTKRMGGVDHFDHIKGTYSVGRRSKRWWLRIFYFIFDACITNSFLLQGKNANATKLSNLEYRVALARGLIGCFSSRKRRAEGVNYVVRKKVAVSENYQKAIHIVAPEIRFSNVGDHMPNDIPSYQRCRYCSTKAKDKRSKIKCSKCGVPLCITPCFSNFHKQV

SEQ ID NO: 3 - DNA, 2166 bp, Artificial sequence
  PLE-wu transposase nucleotide sequence
atgaaaatacttagagatgacgatattgagaaggagttgaatggtatgttcggtttgtcagaaccggaagagtctgaagacggcgaagaatcagaagacgatgacgttccgtacaatgtcactaagttacagcgaattttaggggatttagaagaaccagaccttcgatctactgcaacaattcgagcatcgttatcgcctcaactatcaacatcatctgatgcggagatcccatgcggtgagcctttacttgtaatacctccacctactgtgatagaagctgttgcgtctacgtctgcagtgccattacatgtgccgcaaccagctgtactactgcctgctgagcctcctatttcacgtaagttgcctgtaaaaggtagaggacgaggagctaggatggaacgtggatccggaacgggacgtagatttagaacaggacgagaagctggaactagaatgggacgtggagctagaacgaggctaggaagtggggttggagataaagacagcgattcatccgaagataatacgccaccagtatcagaagcagagtcagaagcaagcgattctgacagagatgaagaagagtggaaaaaaaccttgtggactgatattaggccacagcttgagtcctttgatagtgttccaatgactccgacacgcgtgttaccatctaatgccaggcctatccgatattttgaaaaattttttactcaagaagtttttgaattaattatcacagagactaacagatatgcatgtcaaaacaacgtaataggctggaccatactagacataaaggaattgaaggctttcctaggtatattgattattatgggatacaatattttacctactttcgaattgtattggtcatcggatcccgaatttagagttgacgaaatagcttccaccatgacattccgaagattcaagcaaattttacgttgtttgcatctaaatgataattctaaacaacctgccagacttagccccgagtatgataagctattcaaaattcgcccgttattgacgcttataaacacctcttttcaagaaaatgcgcacaactcttcctctcagtcaatagatgaatcaatgattcttttcaaaggaaggtctacattgaagcaatacatgcctatgaagccaataaaaaggggtttcaaggtatggtgccgctgtgatagtataactggttatctgtacgaatttgatatatacaccggaagagatggtgacagagtagaagacaatttaggaggaaaagtggtcaaaaagttaacagaaaaattgaaaggaatggcggcagtgcatgttacatttgataattttttttgcagttatgatatcatgaattatctacatgtaaatggtatttctgcgagcggtactgtacgcaggcaaagagcagacttaccgaaacttgtgaaaagtaaaaaaaaactaaaactaacaaaagggcagtacaaatggagagtgaaggagaacgtggcttttgtaatctggcaagatacaaaagaagtcttatttatgaccaatgctttccatcccaaggacaatgaaacatctctacccaggaaaggaagagacggttccaagacagatgtaaggtgtcctgcagtagtgaaagaatatacaaaaagaatgggtggagtagatcactttgatcacatcaaagggacatactcagtggggcgaaggagcaaacggtggtggcttcgcatattttattttatttttgatgcctgcatcacaaattcttttttactccagggaaagaatgcgaatgcaaccaaattgtccaacctagagtatcgtgtcgctctagccagaggacttattggttgtttttcttcccgaaaacgtcgtgctgaaggcgtcaattacgtcgtaagaaaaaaagttgctgtatctgaaaattatcaaaaggcaattcacatcgttgcaccggaaattcgcttttccaacgttggtgatcacatgccaaatgatataccgtcttatcaaagatgccgatattgcagcactaaggcaaaagataagcgatctaagatcaagtgtagcaaatgtggagtaccgttatgtataacaccatgcttttcgaattttcacaaacaagtttaattttcttttatttttgtttctattttcttattggtattc

SEQ ID NO: 4 - DNA, 379 bp, Artificial sequence
  PLE-wu 5′ end
ttaacccttttgcagttagagggaatatttttcccaccaataaaaaccaataaaaaaggaacaaaatttctatttgcaattttatttttcgacctgacaacattaacgtaacattatgacatttgtttctgattttatttgacattgacagattcaattttttttcaaagtcttgaggtgaatttgaaaactagcggttttttgtacttcgagagaaatcggtacatatctttgcaatagagtggtgaaaaaaggtaagttcttctagatgttgtgcttttgaatgttttttcatagatataagacgtattttttatgtatctgccacgaattttcatatatgtcgattttttttggtgggaagaatattccctctaac

SEQ ID NO: 5 - DNA, 148 bp, Artificial Sequence
  PLE-wu 3′ end
ggtagagggaatattcttaccaccaattaaaaaaaaaagtttaaaaaaaattgaatctcaatttttattttaggcatcttatgtaacacaaatccgaaaaccaaatctttctatctcatctagatccttataaccgttaaagggttaa

SEQ ID NO: 6 - DNA, 27 bp, Artificial sequence
  Forward primer for amplifying PLE-wu transposon
gcggaattcgcaacagagcgtcgcgag

SEQ ID NO: 7 - DNA, 29 bp, Artificial sequence
  Reverse primer for amplifying PLE-wu transposon
gcggaattcagcggagcaaaagctgttac

SEQ ID NO: 8 - DNA, 37 bp, Artificial sequence
  Forward primer for amplifying 708-residue PLE-wu transposase
cgcgaattcgccaccatgaaaatacttagagatgacg

SEQ ID NO: 9 - DNA, 37 bp, Artificial sequence; identical to SEQ ID NO: 8
  Reverse primer for amplifying 708-residue PLE-wu transposase

SEQ ID NO: 10 - DNA, 29 bp, Artificial sequence
  Forward primer for amplifying Full-5′ end of PLE-wu
gctgaattcaacacgcgactcttttcgtc

SEQ ID NO: 11 - DNA, 27 bp, Artificial sequence
  Reverse primer for amplifying Full-5′ end of PLE-wu (1)
gcgactagtagaaagcggatacttcac

SEQ ID NO: 12 - DNA, 32 bp, Artificial sequence
  Forward primer for amplifying Full-3′ end of PLE-wu
gcgacgcgtattggtattcaattgtgtgattg

SEQ ID NO: 13 - DNA, 29 bp, Artificial sequence
  Reverse primer for amplifying Full-3′ end of PLE-wu
gctgaattcaagaatggcaaaccaagtcg

SEQ ID NO: 14 - DNA, 29 bp, Artificial sequence
  Forward primer for checking for excision of PLE-wu (P1)
cgcctcgaggacgtctggcaagaatcaca

SEQ ID NO: 15 - DNA, 29 bp, Artificial sequence
  Reverse primer for checking for excision of PLE-wu (P2)
cgcaagcttcacgacagcaggctgaataa

SEQ ID NO: 16 - DNA, 20 bp, Artificial sequence
  Forward primer for checking for excision of PLE-wu (P3)
gaaccgtcagatccgctagt

SEQ ID NO: 17 - DNA, 20 bp, Artificial sequence
  Reverse primer for checking for excision of PLE-wu (P4)
gaacttcagggtcagcttgc

SEQ ID NO: 18 - DNA, 35 bp, Artificial sequence
  Forward primer for amplifying 576-residue transposase
cgcgaattcgccaccatggaacgtggatccggaac

SEQ ID NO: 19 - DNA, 32 bp, Artificial sequence
  Reverse primer for amplifying 576-residue transposase
gcggaattcttaaacttgtttgtgaaaattcg

SEQ ID NO: 20 - DNA, 30 bp, Artificial sequence
  Reverse primer for amplifying Full-5′ end of PLE-wu (2)
gcgacgcgtagtagaaagcggatacttcac

SEQ ID NO: 21 - DNA, 29 bp, Artificial sequence
  Forward primer for amplifying deleted-5′ end of PLE-wu (i.e. short PLE-wu/short Tpase)
gcgacgcgttacgttaatgttgtcaggtc

SEQ ID NO: 22 - DNA, 31 bp, Artificial sequence
  Forward primer for amplifying deleted-3′ end of PLE-wu (i.e. short PLE-wu/short Tpase)
gcgacgcgtgcatcttatgtaacacaaatcc

SEQ ID NO: 23 - DNA, 20 bp, Artificial sequence
  Reverse primer for amplifying 3′end of PLE-wu and its nearby chromosome region
tgaacccaccacatttcaga

SEQ ID NO: 24 - DNA, 112 bp, Artificial sequence
  5′ end of short/ truncated PLE-wu transposon
ttaacccttttgcagttagagggaatatttttcccaccaataaaaaccaataaaaaaggaacaaaatttctatttgcaattttatttttcgacctgacaacattaacgtaac

SEQ ID NO: 25 - DNA, 74 bp, Artificial sequence
  3′ end of truncated / short PLE-wu transposon
catcttatgtaacacaaatccgaaaaccaaatctttctatctcatctagatccttataaccgttaaagggttaa

SEQ ID NO: 26 - DNA, 21 bp, Artificial sequence
  Forward primer for amplifying 3′end of PLE-wu and its nearby chromosome region
cacaaatccgaaaaccaaatc

SEQ ID NO: 27 - protein, 576 aa, Artificial sequence
  PLE-wu short transposase
MERGSGTGRRFRTGREAGTRMGRGARTRLGSGVGDKDSDSSEDNTPPVSEAESEASDSDRDEEEWKKTLWTDIRPQLESFDSVPMTPTRVLPSNARPIRYFEKFFTQEVFELIITETNRYACQNNVIGWTILDIKELKAFLGILIIMGYNILPTFELYWSSDPEFRVDEIASTMTFRRFKQILRCLHLNDNSKQPARLSPEYDKLFKIRPLLTLINTSFQENAHNSSSQSIDESMILFKGRSTLKQYMPMKPIKRGFKVWCRCDSITGYLYEFDIYTGRDGDRVEDNLGGKVVKKLTEKLKGMAAVHVTFDNFFCSYDIMNYLHVNGISASGTVRRQRADLPKLVKSKKKLKLTKGQYKWRVKENVAFVIWQDTKEVLFMTNAFHPKDNETSLPRKGRDGSKTDVRCPAVVKEYTKRMGGVDHFDHIKGTYSVGRRSKRWWLRIFYFIFDACITNSFLLQGKNANATKLSNLEYRVALARGLIGCFSSRKRRAEGVNYVVRKKVAVSENYQKAIHIVAPEIRFSNVGDHMPNDIPSYQRCRYCSTKAKDKRSKIKCSKCGVPLCITPCFSNFHKQV

SEQ ID NO: 28 - DNA, 1731 bp, Artificial sequence
  PLE-wu nucleotide sequence encoding short transposase
atggaacgtggatccggaacgggacgtagatttagaacaggacgagaagctggaactagaatgggacgtggagctagaacgaggctaggaagtggggttggagataaagacagcgattcatccgaagataatacgccaccagtatcagaagcagagtcagaagcaagcgattctgacagagatgaagaagagtggaaaaaaaccttgtggactgatattaggccacagcttgagtcctttgatagtgttccaatgactccgacacgcgtgttaccatctaatgccaggcctatccgatattttgaaaaattttttactcaagaagtttttgaattaattatcacagagactaacagatatgcatgtcaaaacaacgtaataggctggaccatactagacataaaggaattgaaggctttcctaggtatattgattattatgggatacaatattttacctactttcgaattgtattggtcatcggatcccgaatttagagttgacgaaatagcttccaccatgacattccgaagattcaagcaaattttacgttgtttgcatctaaatgataattctaaacaacctgccagacttagccccgagtatgataagctattcaaaattcgcccgttattgacgcttataaacacctcttttcaagaaaatgcgcacaactcttcctctcagtcaatagatgaatcaatgattcttttcaaaggaaggtctacattgaagcaatacatgcctatgaagccaataaaaaggggtttcaaggtatggtgccgctgtgatagtataactggttatctgtacgaatttgatatatacaccggaagagatggtgacagagtagaagacaatttaggaggaaaagtggtcaaaaagttaacagaaaaattgaaaggaatggcggcagtgcatgttacatttgataattttttttgcagttatgatatcatgaattatctacatgtaaatggtatttctgcgagcggtactgtacgcaggcaaagagcagacttaccgaaacttgtgaaaagtaaaaaaaaactaaaactaacaaaagggcagtacaaatggagagtgaaggagaacgtggcttttgtaatctggcaagatacaaaagaagtcttatttatgaccaatgctttccatcccaaggacaatgaaacatctctacccaggaaaggaagagacggttccaagacagatgtaaggtgtcctgcagtagtgaaagaatatacaaaaagaatgggtggagtagatcactttgatcacatcaaagggacatactcagtggggcgaaggagcaaacggtggtggcttcgcatattttattttatttttgatgcctgcatcacaaattcttttttactccagggaaagaatgcgaatgcaaccaaattgtccaacctagagtatcgtgtcgctctagccagaggacttattggttgtttttcttcccgaaaacgtcgtgctgaaggcgtcaattacgtcgtaagaaaaaaagttgctgtatctgaaaattatcaaaaggcaattcacatcgttgcaccggaaattcgcttttccaacgttggtgatcacatgccaaatgatataccgtcttatcaaagatgccgatattgcagcactaaggcaaaagataagcgatctaagatcaagtgtagcaaatgtggagtaccgttatgtataacaccatgcttttcgaattttcacaaacaagtttaa

SEQ ID NO: 29 - DNA, 20 bp, Artificial sequence
  5′ end PLE-wu imperfect inverted repeat
cccttttgcagttagaggga

SEQ ID NO: 30 - DNA, 20 bp, Artificial sequence
  3′ end PLE-wu imperfect inverted repeat
tccttataaccgttaaaggg

SEQ ID NO: 31 - DNA, 6 bp, Artificial sequence
  5′ end of PLE-wu perfect inverted terminal repeat
cccttt

SEQ ID NO: 32 - DNA, 6 bp, Artificial sequence
  3′ end of PLE-wu perfect inverted terminal repeats
aaaggg

SEQ ID NO: 33 - DNA, 32 bp, Artificial sequence
  Sub-terminal repeat 1
gttagagggaatatttttcccaccaataaaaa

SEQ ID NO: 34 - DNA, 32 bp, Artificial sequence
  Sub-terminal repeat 2
ttttttttggtgggaagaatattccctctaac

SEQ ID NO: 35 - DNA, 32 bp, Artificial sequence
  Sub-terminal repeat 3
ggtagagggaatattcttaccaccaattaaaa

(19) United States
(12) Patent Application Publication
(10) Pub. No.: US 2017/0159070 A1
(43) Pub. Date: Jun. 8, 2017

(54) TRANSPOSON FOR GENOME MANIPULATION

(71) Applicant: AGENCY FOR SCIENCE, TECHNOLOGY AND RESEARCH, Singapore (SG)

(72) Inventor: Chunxiao WU, Singapore (SG)

(21) Appl. No.: 15/316,508

(22) PCT Filed: Jun. 6, 2014
(86) PCT No.: PCT/SG2014/000261

§ 371 Date: Dec. 5, 2016

(30) Foreign Application Priority Data
Jun. 6, 2013 (SG) 201304405-2

(51) Int. Cl. C12N 15/85; C12N 9/12; C12N 15/11

(52) U.S. Cl. CPC C12N 15/85; C12N 15/11; C12N 15/111; C12N 9/1241; C12N 2303/00; C12N 2830/48; C12N 2800/90

(57) ABSTRACT

Disclosed is a vector comprising a piggyBac like element (PLE) transposon and/or transposase. Also disclosed are vectors comprising a combination of the vectors of the present disclosure, a transformation system comprising the vectors of the present disclosure, an oligonucleotide primer, methods of amplifying nucleotide sequences, methods of transforming a cell with a gene and a transgenic organism.

### CROSS-REFERENCE TO RELATED APPLICATIONS

This application claims the benefit of priority of Singapore patent application No. 201304405-2, filed 6 Jun. 2013, the contents of it being hereby incorporated by reference in its entirety for all purposes.

### FIELD OF THE INVENTION

The present invention is directed to nucleic acid transformation constructs encoding mobile elements and their use for transforming eukaryotic cells.

### BACKGROUND OF THE INVENTION

Transposons are genetic elements that have the ability to move from one location to another within a genome. They can be divided into two classes, Class I and Class II transposons. Class I transposons, also known as retrotransposons, use reverse transcriptase to make new DNA copies from RNA and insert the DNAs into new locations. Class II transposons do not have the RNA intermediate step, instead they use “cut and paste” mechanism, directly excising out the DNA fragment and inserting it to a new location in the genome.

piggyBac (IFP2), originally identified from a mutant baculovirus cultured in Trichoplusia ni insect line TN-368, is a well-studied Class II transposon. It produces TTAA target site duplication at the junctions of new location and restores the original TTAA sequence upon excision (traceless excision), leaving no footprint behind. Due to its high transposition activity in different species of insects, it has become the most widely used system for germ-line transformation, as well as genetic tools for gene tagging and enhancer trapping. Interestingly, piggyBac is also functional in mammalian cells and has the highest activity among all known vertebrate transposons. Thus, it has quickly become a powerful tool for genome manipulation, including gene trapping, creation of transgenic animals, and as nonviral gene therapy vectors. Recently it has also been applied to generate transgene-free induced pluripotent stem (iPS) cells, owing to its unique traceless excision characteristic.

In recent years, a wide variety of piggyBac-like elements (PLE) from other insect species have been identified, either by searching for sequences similar to piggyBac in the genome or from mutant baculovirus isolation. In vertebrates, sequences with similarity to PLE have also been found. A new member of PLE, TxpB, has been identified from Xenopus species. Among these newly identified PLEs, Uribo2 from Xenopus tropicalis and AgoPLE1.1 from Aphis gossypii show excision activity. piggybat, a new member of PLE found in the little brown bat (Myotis lucifugus), has transposition activity in human, bat and yeast cells. However, the transposition rates of transposon in mammalian cells are quite low. Among Tol2, Sleeping Beauty and piggyBac, the three commonly used transposon system, piggyBac shows the highest activity. To improve the transposition rate, various point mutations have been introduced into the original transposase. However, testing various point mutations to derive a high activity transposase is not cost or time effective.

In view of the increasing demand for a powerful tool for gene manipulation, there is a need to provide alternative transposon and transposase. Thus, there is a need to provide alternative transposon and transposase that can also have activity in mammals.

### SUMMARY OF THE INVENTION

In a first aspect, there is provided a vector, comprising a piggyBac like element (PLE) transposon.

In a second aspect, there is provided a vector, comprising a nucleotide sequence encoding a piggyBac like element (PLE) transposase.

In a third aspect, there is provided a vector according to the first aspect, to be used in combination with a vector according to the second aspect.

In a fourth aspect, there is provided a transformation system of the present disclosure. The transformation system comprises (a) a first vector according to the first aspect, wherein the first vector comprises a PLE transposons. The transformation system may further comprise a second vector according to the second aspect, wherein the second vector comprises a nucleotide sequence that encodes a PLE transposase. In another embodiment of the fourth aspect, the transformation system may be a single vector comprising the PLE transposon as defined in the first aspect and the nucleotide sequence that encodes the PLE transposase as defined in the second aspect.

In a fifth aspect, there is provided a transformation system comprising a first vector according to the first aspect, wherein the first vector comprises a PLE transposon; and a nucleic acid encoding a transposase according to the second aspect or a transposase protein for delivery with the first vector.

In a sixth aspect, there is provided a kit for transforming a cell comprising one or more vectors according to the present disclosure.

In a seventh aspect, there is provided a kit for transforming a cell comprising a single vector as defined herein.

In an eight aspect, there is provided an oligonucleotide primer comprising the nucleotide sequence of any one of SEQ ID NOs: 6 to 23.

In a ninth aspect, there is provided a method of amplifying a nucleotide sequence of SEQ ID NO: 1. The method of this aspect comprises performing a polymerase chain reaction using a forward primer comprising the nucleotide sequence of SEQ ID NO: 6 and a reverse primer comprising the nucleotide sequence of SEQ ID NO: 7.

In a tenth aspect, there is provided a method of amplifying a nucleotide sequence of SEQ ID NO: 3. The method of this aspect comprises performing a polymerase chain reaction using a forward primer comprising nucleotide sequence of SEQ ID NO: 8 and a reverse primer comprising nucleotide sequence of SEQ ID NO: 9.

In an eleventh aspect, there is provided a method of amplifying a nucleotide sequence of SEQ ID NO: 4. The method of this aspect comprising performing a polymerase chain reaction using a forward primer comprising nucleotide sequence of SEQ ID NO: 10 and a reverse primer comprising nucleotide sequence of SEQ ID NO: 11 or SEQ ID NO: 20.

In a twelfth aspect, there is provided a method of amplifying nucleotide sequence of SEQ ID NO: 5. The method of this aspect comprising performing a polymerase chain reaction using a forward primer comprising nucleotide sequence of SEQ ID NO: 12 and a reverse primer comprising nucleotide sequence of SEQ ID NO: 13.

In a thirteenth aspect, there is provided a method of transforming a cell into a gene. The method of this aspect comprises (a) transfecting the cell with a first vector according to the first aspect, wherein the first vector comprises the 5′ and 3′ ends of a PLE transposon; and transfecting the cell with a second vector according to the second aspect, wherein the second vector comprises a nucleotide sequence that encodes a PLE transposase. The method of this aspect may alternatively comprise (b) transfecting the cell with a single vector, wherein the single vector comprises the 5′ and 3′ ends of the PLE transposon and the nucleotide sequence that encodes the PLE transposase.

In a fourteenth aspect, there is provided a method of transforming a cell with a gene. In the method of this aspect, the method comprises transfecting the cell with a first vector according to the first aspect, wherein the first vector comprises the 5′ and 3′ end of a PLE transposon; and delivering a nucleic acid encoding a transposase as defined in the second aspect or a transposase protein either together with the first vector or before transfection of the first vector or after transfection of the first vector.

In a fifteenth aspect, there is provided a method of transforming a cell with a gene. In the method of this aspect, the method comprises delivering a nucleic acid encoding a transposase as defined in the second aspect, or a transposase protein into the cell, wherein the cell already comprises the 5′ and 3′ ends of a PLE transposon.

In a sixteenth aspect, there is provided a transgenic organism. The transgenic organism comprises cells which have been transformed by the methods of the present disclosure.

### BRIEF DESCRIPTION OF THE DRAWINGS

The invention will be better understood with reference to the detailed description when considered in conjunction with the non-limiting examples and the accompanying drawings, in which:

FIG. 1 shows the nucleotide sequence of PLE-wu transposon (SEQ ID NO: 1) and the amino acid sequence of the putative transposase (SEQ ID NO: 2). The duplicated TTAA target sequence on both ends of PLE-wu is shown in bold. The 6-bp inverted terminal repeats next to the TTAA target site are indicated by arrows below. The 32-bp imperfect sub-terminal direct and inverted repeats are indicated by arrows with the 13-bp stretch of perfect match shown as bold letters. The putative polyadenylation signal is shown in italics. The start codon, for the short form of transposase is underlined and shown in bold. Thus, FIG. 1 shows an exemplary transposons and transposase of the present disclosure. FIG. 1 also shows the short or truncated transposase (576 residues).

FIG. 2 shows nucleotide sequences of the junction of the PLE-wu insertion site on Baculovirus (fp25K) and U87 human glioma cells (lipase H). The duplicated TTAA sites are shown in bold. FIG. 2 illustrates typical characteristics of piggyBac-like element transposition event, which shows insertion results in duplication of TTAA at the junctions.

FIG. 3 shows gel electrophoresis results of traceless excision of PLE-wu from the disrupted fp25K gene in mutant baculovirus. Primers P1 and P2 used in the PCR reaction are shown as dotted lines with arrowheads. The two PCR products with different size are indicated by the two arrowheads with solid lines. The sequence of the restored fp25K gene is shown underneath the diagram of excision event. FIG. 3 shows PLE-wu is tracelessly excised from the final product.

FIG. 4 (A) shows diagram showing the excision of PLE-wu transposon from the EGFP expression cassette (pCMVpPH_PLE-wu_EGFP). pCMV, CMV promoter; pPH, polyhedron promoter; pA, polyadenylation signal. Primers. P3 and P4 used in the PCR reaction are shown as dotted lines with arrowheads. The arrowheads with solid line indicate the translation start sites for the transposase of PLE-wu and EGFP; FIG. 4 (B) shows phase contrast and fluorescent image of baculovirus plaque formed on Sf9 cells, transfected 4T1 mouse mammary tumor cells, and U87 humane glioma cells. Only a few green cells (seen as bright dots in black background) were observed on the second day after transfection. FIG. 4 (C) shows. PCR results of genomic DNAs using primers P3 and P4. −, un-transfected cells; +, cells transfected with pCMV_pPH_PLE-wu_EGFP; M, molecular weight marker. FIG. 4 (D) shows sequencing result of the PCR product as indicated by the arrow in FIG. 4 (C). The restored TTAA insertion target site is boxed. Thus, FIG. 4 shows traceless excision activity of PLE-wu in both insect and mammalian cells.

FIG. 5 (A) provides a schematic presentation of the donor and helper plasmids. pRSV, RSV promoter; pCMV, CMV promoter; pA, polyadenylation signal; pSV40, SV40 promoter; Neo, neomycin resistant gene; Amp, ampicillin resistant gene; ColE1, origin of plasmid replication; BamHI, BamHI restriction site; pPH, polyhedrin promoter; WPRE, Woodchuck hepatitis virus posttranscriptional regulatory element; RU5, R segment and part of the U5 sequence of the HTLV Type 1 Long Terminal Repeat; PLE-wu-Tpase, the 708-residue transposase of PLE-wu. The 3′ and 5′ part of PLE-wu are shown as big solid arrowheads. The small arrowheads with solid lines indicate the translation initiation sites for EGFP, Neo, and the transposase of PLE-wu. FIG. 5 (B) shows the results of colony formation assays. U87 human glioma cells were co-transfected with donor and helper plasmids. The G418 resistant colonies were stained and counted after two weeks. Data represent mean values with standard deviations of three experiments. Accordingly, FIG. 5 shows transposition activity of PLE-wu in mammalian cells. Thus, showing the vectors of the present disclosure are active in mammalian cells.

FIG. 6 (A) provides a schematic presentation of the deletion at 5′ and 3′ end of PLE-wu. The truncated PLE-wu that remained after internal deletions at the 5′ and 3′ end of PLE-wu used in the donor plasmids are indicated by arrowheads with solid lines (SEQ ID NO: 23 for 5′ end of truncated PLE-wu transposon and SEQ ID NO: 24 for 3′ end of truncated PLE-wu transposon). The underlined inverted terminal repeats are perfect match, with the 5′ end (cccttt) and 3′ end (aaaggg) complement each other. The sub-terminal repeats are also underlined. The imperfect inverted repeats are provided in the dashed boxes and labelled SEQ ID NOs: 29 and 30. The start codons for 708-residue-long (SEQ ID NO: 1) and short form of transposase (SEQ ID NO: 27) used in the helper plasmids are indicated by the arrowheads; FIG. 6 (B) shows the results of colony formation assays. U87 human glioma cells were co-transfected with donor and helper plasmids. The G418 resistant colonies were stained and counted after two weeks. Data represent mean values with standard deviations of three experiments. Thus, FIG. 6 shows the transposition activity of PLE-wu with deletions on terminal sequences.

### DETAILED DESCRIPTION OF THE PRESENT INVENTION

The inventors of the present disclosure found a new piggyBac-like element (PLE) transposon and transposase, named PLE-wu transposon and transposase from a mutant baculovirus cultured in Sf9 insect cells. As would be appreciated in the art, a transposon usually contains two parts: an open reading frame that encodes transposase at the middle of transposon and terminal sequences at 5′ and 3′ end of the transposon. The translated transposase binds to the 5′ and 3′ sequence of the transposon and carries out the transposition function. piggyBac, a highly active transposon in insect and mammalian cells, is a very useful tool in genome manipulation. Thus, it is envisaged that the new piggyBac-like element (PLE) transposon may be a useful alternative transposon. As used herein, the term “transposon” may be used interchangeably with transposable elements, which are used to refer to polynucleotides capable of inserting copies of themselves into other polynucleotides. The term transposon is well known to those skilled in the art and includes classes of transposons that can be distinguished on the basis of sequence organization, for example short inverted repeats at each end, and/or directly repeated long terminal repeats (LTRs) at the ends. The transposon as described herein may be described as a “piggyBac like” element, which phrase is used to refer to transposon element that is characterized by its traceless excision, which recognizes TTAA sequence and restores the sequence at the insert site back to the original TTAA sequence after removal of the transposon. Thus, in a first aspect, there is provided a vector, comprising a piggyBac like element (PLE) transposon. As used herein, the term “vector” refers to a linear or circular DNA molecule that comprises a polynucleotide encoding a polypeptide and is operably linked to control sequences, wherein the control sequences provide for expression of the polynucleotide encoding the polypeptide. At a minimum, the expression vector comprises a promoter sequence, and transcriptional and translational stop signal sequences. Such vectors may include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cos-mids and phagemids. The expression system constructs may contain control regions that regulate as well as engender expression. Generally, any system or vector suitable to maintain, propagate or express polynucleotides and to synthesise a polypeptide in a host may be used for expression in this regard. The appropriate DNA sequence may be inserted into the expression system by any of a variety of well-known and routine techniques.

Accordingly, in one example, the vector as described herein may comprise at least one pair of an inverted terminal repeat at the 5′ and 3′ ends of the transposon. As used herein, the term “inverted terminal repeat” refers to a sequence located at one end of a vector that can form a hairpin structure when used in combination with a complementary sequence that is located at the opposing end of the vector. The pair of inverted terminal repeats is involved in the transposition activity of the transposon of the vector of the present disclosure, in particular involved in DNA addition or removal and excision of DNA of interest. In one example, at least one pair of an inverted terminal repeat appears to be the minimum sequence required for transposition activity in a plasmid. In another example, the vector of the present disclosure may comprise at least two, three or four pairs of inverted terminal repeats. As would be understood by the person skilled in the art, to facilitate ease of cloning, the necessary terminal sequence may be as short as possible and thus contain as little inverted repeats as possible. Thus, in one example, the vector of the present disclosure may comprise not more than one, not more than two, not more than three or not more than four pairs of inverted terminal repeats. In one example, the vector of the present disclosure may comprise only one inverted terminal repeat. Whilst not wishing to be bound by theory, it is envisaged that having more than one inverted terminal repeat may be disadvantageous as it may lead to non-specific transposase binding to the multiple inverted terminal repeats and resulting in the removal of desired sequence or insertion of undesirable sequences. The inverted terminal repeat of the present disclosure may form either a perfect inverted terminal repeat (or interchangeably referred to as “perfect inverted repeat”) or imperfect inverted terminal repeat (or interchangeably referred to as “imperfect inverted repeat”). As used herein, the term “perfect inverted repeat” refers to two identical DNA sequences placed at opposite direction. In one example, a perfect inverted repeat may include, but is not limited to, cccttt (SEQ ID NQ: 31) and aaaggg (SEQ ID NO: 32), which may act as the binding site for the transposase. In contrast, the term “imperfect inverted repeat” refers to two DNA sequences that are similar to one another except that they contain a few mismatches. These repeats (i.e. both perfect inverted repeat and imperfect inverted repeat) are the binding sites of transposase. In one example, the imperfect inverted repeat may include, but is not limited to the sequence cccttttgcagttagaggga (SEQ ID NO: 29) and tccttataaccgttaaaggg (SEQ ID NO: 30).

In one example, the transposon may have an asymmetric terminal structure, including, but is not limited to inverted terminal repeats, imperfect inverted repeats and sub-terminal repeats. Accordingly, the vector as described herein may further comprise at least one, two, three or more pairs of sub-terminal inverted repeat. As used herein the term “sub-terminal inverted repeat” refers to complementary sequences of DNA that repeat at least once, twice, or more times and are found at either end of the transposon of the present disclosure. These sub-terminal inverted repeat may be involved in transposon's activity in inserting or removing genetic material from the host genome. Unlike the inverted terminal repeats, sub-terminal inverted repeats may be excluded from the vector of the present disclosure. In one example, the sub-terminal inverted repeat may be the sequence, gttagagggaatattMcccaccaataaaaa (SEQ ID NO: 33), ttttttttggtgggaagaatattccctctaac (SEQ ID NO: 34), ggtagagggaatattcttaccaccaattaaaa (SEQ ID NO: 35), and the like. In one example, the vector of the present disclosure may comprise three sub-terminal inverted repeats, which may be useful for the binding of the untruncated 708-residue transposase, but not for the truncated or short form of the transposase (i.e. the 576-residue transposase). The sub-terminal inverted repeats may act as the binding site for the extra-residues at the N-terminal of 708-residue transposase. Without wishing to be bound by theory, it is envisaged that an increase number of copies of sub-terminal inverted repeats may enhance the activity of the 708-residue transposase. The 708-residue transposase binds to both inverted terminal repeats and sub-terminal repeats. However, sub-terminal repeats may not replace inverted terminal repeats. Upon binding to the inverted terminal repeats, transposase will cut out the DNA between the inverted terminal repeats. The 708-residue transposase extra residues at the N-terminal binds to sub-terminal repeats, while the rest of the transposase, which includes the short form of transposase, binds to the inverted terminal repeats.

In one example, the transposition activity of vector of the present disclosure may be host factor-independent. In one example, the 5′ and 3′ ends of the transposon of the present disclosure may be used to flank a gene, which is to be transformed into a cell. As the name of this transposon illustrates, the PLE transposon may not be a piggyBac transposon.

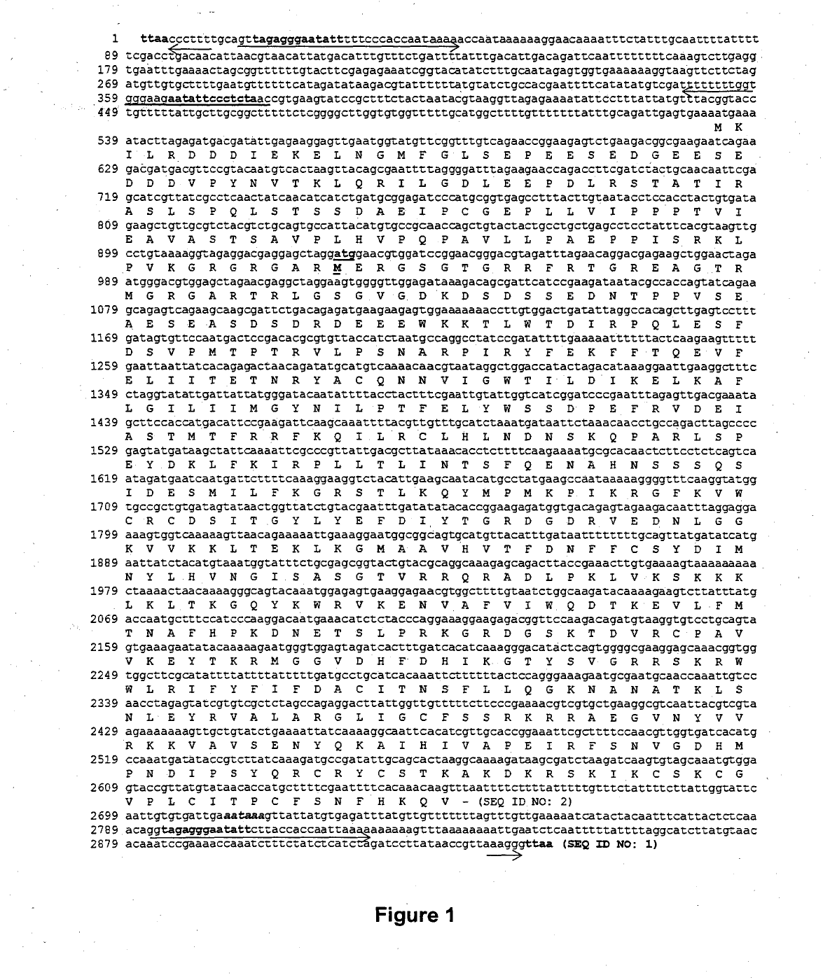

Accordingly, in one example, the PLE transposon may comprise SEQ ID NO: 1; or a sequence with at least 70%, or at least 75%, or at least 80%, or at least 85%, or at least 90% or at least 95%, or at least 97.5%, or at least 98%, or at least 99% identity to SEQ ID NO: 1, which is shown FIG. 1 to be 2931 bp in length. The sequence of PLE-wu has been submitted to the DDBJ/EMBL/GenBank databases under accession number AB841319.

As illustrated in FIG. 6, the transposon of the present disclosure has also been found to be active even when either the 5′ or 3′ ends of the transposon is deleted. Thus, in one example, the transposon may be deleted at the 5′ and/or 3′ ends of the transposon. In another example, the 5′ and/or 3′ end deletions may be an internal deletion. That is, the deletion may occur not necessarily at the exact 5′ and/or 3′ ends of the transposon. In one example, the 5′ and/or 3′ end deletion removes the sub-terminal repeats of a transposon. It was found that the sub-terminal repeats of a transposon are not critical in transposition function. In one example, the deletion at 3′ end may result in the 3′ end having no sub-terminal repeat. In another example, the deletion at 5′ end may result in the 5′ end having none or one sub-terminal repeat. Accordingly, in one example, the PLE transposon may not comprise any sub-terminal repeats.

As illustrated by FIG. 6, the vector as described herein may comprise a deletion of about 1 bp to about 250 bp, about 50 bp to about 225 bp, or about 201 bp of the nucleotide sequence encoding the 5′ end of the SEQ ID NO: 4 (PLE-wu 5′ end). In one example, the vector as described herein may comprise a deletion of about 1 bp to about 100 bp, about 50 bp to about 80 bp, or about 74 bp of the nucleotide sequence encoding the 3′ end of the SEQ ID NO: 5 (PLE-wu 3′ end). Accordingly, in one example, the vector as described herein may comprise SEQ ID NO: 4 (PLE-wu 5′ end) or SEQ ID NO: 24 (5′ end of truncated/short PLE-wu); or at least a sequence with at least 50%, at least 60%, at least 70%, or at least 80%, or at least 85%, or at least 90%, or at least 95%, or at least 97.5%, or at least 98%, or at least 99% identity to SEQ ID NO: 4 (PLE-wu 5′ end) or SEQ ID NO: 24 (5′ end of truncated/short PLE-wu); and/or SEQ ID NO: 5 (PLE-wu 3′ end) or SEQ ID NO: 25 (3′ end of truncated/short PLE-wu). In another example, the vector as described herein may comprise SEQ ID NO: 5 (PLE-wu 3′ end) or SEQ ID NO: 25 (3′ end of truncated/short PLE-wu); or at least a sequence with at least 50%, at least 60%, at least 70%, or at least 80%, or at least 85%, or at least 90%, or at least 95%, or at least 97.5%, or at least 98%, or at least 99% identity to SEQ ID NO: 5 (PLE-wu 3′ end) and/or SEQ ID NO: 4 (PLE-wu 5′ end); or SEQ ID NO: 24 (5′ end of truncated/short PLE-wu) and/or SEQ ID NO: 25 (3′ end of truncated/short PLE-wu).

In one example, the 5′ end of a PLE-wu transposon may comprise the following sequence: (SEQ ID NO: 4):

| | |
|---|---|
| ttaacccttt tgcagttaga gggaatattt ttcccaccaa taaaaaccaa taaaaaagga | 60 |
| acaaaatttc tatttgcaat tttatttttc gacctgacaa cattaacgta acattatgac | 120 |
| atttgtttct gattttattt gacattgaca gattcaattt tttttcaaag tcttgaggtg | 180 |
| aatttgaaaa ctagcggttt tttgtacttc gagagaaatc ggtacatatc tttgcaatag | 240 |
| agtggtgaaa aaaggtaagt tcttctagat gttgtgcttt tgaatgtttt ttcatagata | 300 |
| taagacgtat tttttatgta tctgccacga attttcatat atgtcgattt tttttggtgg | 360 |
| gaagaatatt ccctctaac | 379 |

In one example, the 3′ end of a PLE-wu transposon may comprise the following sequence: (SEQ ID NO: 5):

| | |
|---|---|
| ggtagaggga atattcttac caccaattaa aaaaaaaagt ttaaaaaaaa ttgaatctca | 60 |
| atttttattt taggcatctt atgtaacaca aatccgaaaa ccaaatcttt ctatctcatc | 120 |
| tagatcctta taaccgttaa agggttaa | 148 |

In one example, when the 5′ end is truncated, the 5′ end of a short/truncated PLE-wu transposon may comprise the following sequence: (SEQ ID NO: 24):

| | |
|---|---|
| ttaacccttt tgcagttaga gggaatattt ttcccaccaa taaaaaccaa taaaaaagga | 60 |
| acaaaatttc tatttgcaat tttatttttc gacctgacaa cattaacgta ac | 112 |

In one example, when the 3′ end is truncated, the 3′ end of a short/truncated PLE-wu transposon may comprise the following sequence: (SEQ ID NO: 25):

| | |
|---|---|
| catcttatgt aacacaaatc cgaaaaccaa atctttctat ctcatctaga tccttataac | 60 |
| cgttaaggg ttaa | 74 |

For example, the 5′ end of the short/truncated PLE-wu as illustrated in FIG. 6 comprises about 47% of 5′ end of the PLE-wu; the 3′ end of the short/truncated PLE-wu as illustrated in FIG. 6 comprises about 50% of 3′ end of the PLE-wu. As the transposon of the present disclosure is useful in transforming gene into a cell, in one example, the vector of the present disclosure may further comprise a gene to be transformed into a cell.

The transposon of the present disclosure may also contain an active transposase. As used herein, the term “transposase” refers an enzyme that is capable of forming a functional complex with a transposon end-containing composition (e.g., transposons, transposon ends, transposon end compositions, 5′ end of transposon, 3′ end of transposon) and catalyzing insertion or transposition of the transposon end-containing composition into the double-stranded target DNA with which it is incubated in an in vitro transposition reaction. Thus, in a second aspect, there is provided a vector, comprising a piggyBac like element (PLE) transposase. In one example, the transposase may be used to excise a gene flanked by the 5′ and 3′ ends of a PLE transposon and to integrate the gene into the chromosomes of a cell. The transposase of the present disclosure may not be a piggyBac transposase.

In one example, the PLE transposase may include one or more mutations which enhances the activity of the PLE transposase. In one example, the mutation may include point mutation, deletion, substitution or insertion. For example, the point mutation or deletion occurring on the nucleotide sequence encoding the transposase protein may lead to the truncation of the transposase protein (see for example, short transposase in FIGS. 1 and 6). Mutations that may enhance the activity of the transposase may include, but are not limited to, mutations that optimize codon usage for higher protein expression in target organisms, point mutations that enhance the catalytic function of transposase or stabilize the protein at higher temperature (the optimal temperature for insect cell is about 27° C., whereas for mammalian cells is about 37° C.), and mutations that add (or insert) extra DNA binding domains to the N-terminal of transposase, together with corresponding target binding DNA sites as the sub-terminal repeats. Without wishing to be bound by theory, it is believed that since the sub-terminal repeats in the transposons of the present disclosure may not be critical to the short/truncated form of the transposase, in one example of the present disclosure, the sub-terminal repeats may be replaced with other DNA sequences, together with extra DNA binding domains added to the N-terminal of short transposase (for example, Gal4 binding sites and its binding domain). These examples of mutations may be engineered to enhance the activity and specificity of the transposon of the present disclosure.

In the present disclosure, the inventors of the present disclosure surprisingly found that the nucleotide sequence encoding transposase of the present disclosure may be truncated at either the 5′ and/or 3′ ends without loss of activity. Indeed, the truncation of the 5′ end of the nucleotide sequence encoding transposase resulted in an N-terminal truncated transposase that is capable of providing higher transformation rate of genes in a cell (see FIG. 6B). Thus, in one example, the PLE transposase may include a truncation at the 5′ end of the nucleotide sequence encoding the transposase, which results in a truncated transposase which has enhanced activity. Thus, in one example, the PLE transposase may include an N-terminal truncation, which enhances the activity of the PLE transposase. In one example, the truncation may not necessarily be at the 5′ and/or 3′ end of the nucleotide sequence encoding transposase. That is, the truncation may be an internal truncation of the nucleotide sequence encoding the transposase. In one example, the truncation may be a truncation of a region or a portion of about at least 1 to about 400, about 50 to about 360 bp of the nucleotide sequence that encodes the amino acid sequence of SEQ ID NO: 2; or about at least 1 to 350, about 50 to about 150, or about 132 residues deletion of the total number of amino acid in PLE transposase. In one example, the deletion is at the N-terminal of sequence SEQ ID NO: 2.

When the truncation is an internal truncation, the deletion may not result in the deletion of less than or equal to 10 amino acid residues. Thus, in one example, the truncation and/or deletion may be less than or equal to 9, 8, 7, 6, 5, 4, 3, 2 or 1 amino acid residues. Without wishing to be bound by theory, it is believed that an internal truncation that results in more than 10 amino acid residues may result in non-active transposase.

In one example, the PLE transposase may comprise a nucleotide sequence which encodes an amino acid sequence of SEQ ID NO: 2; or SEQ ID NO: 27 or an amino acid sequence with at least 70%, or at least 80%, or at least 85%, or at least 90%, or at least 95%, or at least 97.5%, or at least 98%, or at least 99% identity to SEQ ID NO: 2; or SEQ ID NO: 27. In another example, the PLE transposase may comprise a nucleotide sequence of SEQ ID NO: 3 or SEQ ID NO: 28; or a sequence with at least 70%, or at least 80%, or at least 85%, or at least 90%, or at least 95%, or at least 97.5%, or at least 98%, or at least 99% identity to SEQ ID NO: 3; or SEQ ID NO: 28. For example, as illustrated in FIG. 6, short transposase (SEQ ID NO: 27) is about 81% identity with SEQ ID NO: 2.

In one example, the truncated nucleotide sequence encoding short/truncated transposase may comprise the following sequence:

(SEQ ID NO: 28)
ATGGAACGTGGATCCGGAACGGGACGTAGATTTAGAACAGGACGAGAAG
CTGGAACTAGAATGGGACGTGGAGCTAGAACGAGGCTAGGAAGTGGGGT
TGGAGATAAAGACAGCGATTCATCCGAAGATAATACGCCACCAGTATCA
GAAGCAGAGTCAGAAGCAAGCGATTCTGACAGAGATGAAGAAGAGTGGA
AAAAAACCTTGTGGACTGATATTAGGCCACAGCTTGAGTCCTTTGATAG
TGTTCCAATGACTCCGACACGCGTGTTACCATCTAATGCCAGGCCTATC
CGATATTTTGAAAAATTTTTTACTCAAGAAGTTTTTGAATTAATTATCA
CAGAGACTAACAGATATGCATGTCAAAACAACGTAATAGGCTGGACCAT
ACTAGACATAAAGGAATTGAAGGCTTTCCTAGGTATATTGATTATTATG
GGATACAATATTTTACCTACTTTCGAATTGTATTGGTCATCGGATCCCG
AATTTAGAGTTGACGAAATAGCTTCCACCATGACATTCCGAAGATTCAA
GCAAATTTTACGTTGTTTGCATCTAAATGATAATTCTAAACAACCTGCC
AGACTTAGCCCCGAGTATGATAAGCTATTCAAAATTCGCCCGTTATTGA
CGCTTATAAACACCTCTTTTCAAGAAAATGCGCACAACTCTTCCTCTCA
GTCAATAGATGAATCAATGATTCTTTTCAAAGGAAGGTCTACATTGAAG
CAATACATGCCTATGAAGCCAATAAAAAGGGGTTTCAAGGTATGGTGCC
GCTGTGATAGTATAACTGGTTATCTGTACGAATTTGATATATACACCGG
AAGAGATGGTGACAGAGTAGAAGACAATTTAGGAGGAAAAGTGGTCAAA
AAGTTAACAGAAAAATTGAAAGGAATGGCGGCAGTGCATGTTACATTTG
ATAATTTTTTTTGCAGTTATGATATCATGAATTATCTACATGTAAATGG
TATTTCTGCGAGCGGTACTGTACGCAGGCAAAGAGCAGACTTACCGAAA
CTTGTGAAAAGTAAAAAAAAACTAAAACTAACAAAAGGGCAGTACAAAT
GGAGAGTGAAGGAGAACGTGGCTTTTGTAATCTGGCAAGATACAAAAGA
AGTCTTATTTATGACCAATGCTTTCCATCCCAAGGACAATGAAACATCT
CTACCCAGGAAAGGAAGAGACGGTTCCAAGACAGATGTAAGGTGTCCTG
CAGTAGTGAAAGAATATACAAAAAGAATGGGTGGAGTAGATCACTTTGA
TCACATCAAAGGGACATACTCAGTGGGGCGAAGGAGCAAACGGTGGTGG
CTTCGCATATTTTATTTTATTTTTGATGCCTGCATCACAAATTCTTTTT
TACTCCAGGGAAAGAATGCGAATGCAACCAAATTGTCCAACCTAGAGTA
TCGTGTCGCTCTAGCCAGAGGACTTATTGGTTGTTTTTCTTCCCGAAAA
CGTCGTGCTGAAGGCGTCAATTACGTCGTAAGAAAAAAAGTTGCTGTAT
CTGAAAATTATCAAAAGGCAATTCACATCGTTGCACCGGAAATTCGCTT
TTCCAACGTTGGTGATCACATGCCAAATGATATACCGTCTTATCAAAGA
TGCCGATATTGCAGCACTAAGGCAAAAGATAAGCGATCTAAGATCAAGT
GTAGCAAATGTGGAGTACCGTTATGTATAACACCATGCTTTTCGAATTT
TCACAAACAAGTTTAA

In one example, the truncated transposase protein, or may be interchangeably referred to as short transposase, may comprise the following sequence:

(SEQ ID NO: 27)
MERGSGTGRRFRTGREAGTRMGRGARTRLGSGVGDKDSDSSEDNTPPVSE
AESEASDSDRDEEEWKKTLWTDIRPQLESFDSVPMTPTRVLPSNARPIRY
FEKFFTQEVFELIITETNRYACQNNVIGWTILDIKELKAFLGILIIMGYN
ILPTFELYWSSDPEFRVDEIASTMTFRRFKQILRCLHLNDNSKQPARLSP
EYDKLFKIRPLLTLINTSFQENAHNSSSQSIDESMILFKGRSTLKQYMPM
KPIKRGFKVWCRCDSITGYLYEFDIYTGRDGDRVEDNLGGKVVKKLTEKL
KGMAAVHVTFDNFFCSYDIMNYLHVNGISASGTVRRQRADLPKLVKSKKK
LKLTKGQYKWRVKENVAFVIWQDTKEVLFMTNAFHPKDNETSLPRKGRDG
SKTDVRCPAVVKEYTKRMGGVDHFDHIKGTYSVGRRSKRWWLRIFYFIFD
ACITNSFLLQGKNANATKLSNLEYRVALARGLIGCFSSRKRRAEGVNYVV
RKKVAVSENYQKAIHIVAPEIRFSNVGDHMPNDIPSYQRCRYCSTKAKDK
RSKIKCSKCGVPLCITPCFSNFHKQV.

Without wishing to be bound by theory, it is believed that the transposase without truncation, which has reduced activity and requires extra elements in the terminal, may provide for a safer as the harmful effect from high frequency transposition will be reduced.

It is envisaged that the vectors of the present disclosure may be used in combination with one another. Thus, in another aspect, there is provided a vector according to the first aspect to be used in combination with a vector according to the second aspect. In yet another aspect, there is provided a vector according to the second aspect to be used in combination with a vector according to the first aspect. That is, a vector comprising the transposon of the present disclosure may be used in combination with a vector comprising the transposase of the present disclosure, or vice versa.

In one example, the vectors of the present disclosure may further include a promoter. As used herein, the term “promoter” refers to a polynucleotide sequence that allows transcription of a target gene to which it is operably linked to and regulates the expression of the gene. The promoter includes sequences that are recognized by a RNA polymerase and a transcription initiation site. In order to express a target protein in a particular cell type or a host cell, a suitable functional promoter must be chosen carefully. Thus, in one example, the promoter may include, but is not limited to CMV, polyhedron, SV40, EF-1 alpha, and the like. Furthermore, to assist the selection of cells comprising the vectors of the present disclosure, the vectors as described herein may further include a selection marker. In one example, the selection marker may be fluorescent marker, an antibiotic resistance marker, or the like. When the selection marker is a fluorescent marker, it may include EGFP, red fluorescent protein, yellow fluorescent protein, lacZ, and the like; when the selection marker is an antibiotic resistance marker, it may include Zeocin, Hygromycin, neomycin and the like.

In one example, the vectors of the present disclosure may further comprise a Woodchuck hepatitis virus posttranscriptional regulatory element. The position of either one the Woodchuck hepatitis virus posttranscriptional regulatory element in relation with the vectors of the present disclosure would be apparent to the person skilled in the art. For example, as illustrated in the Example section of the present disclosure (section “PCR and Plasmid construction”), the Woodchuck hepatitis virus posttranscriptional regulatory element may be placed at the end of the multiple cloning site.

In another example, the vectors may further comprise an R segment and U5 sequence of the HTLV Type 1 Long Terminal Repeat. These elements may be used to construct the vectors of the present disclosure. For example, as illustrated in the Example section of the present disclosure (section “PCR and Plasmid construction”), one vector of the present disclosure was constructed by insert CMV promoter and R segment and part of the U5 sequence (RU5) of the HTLV Type 1 Long terminal Repeat downstream of the baculovirus polyhedron promoter (pPH). Thus, in one example, the vectors of the present disclosure may be placed between pPH and Woodchuck hepatitis virus posttranscriptional regulatory element.

As the vectors of the present disclosure may be useful in a transformation system, in another aspect, there is provided a transformation system. The transformation system may be an in vitro transformation system. The transformation system may comprise a) a first vector according to the first aspect, wherein the first vector comprises a PLE transposon; and a second vector according to the second aspect, wherein the second vector comprises a nucleotide sequence that encodes a PLE transposase. Thus, as used herein, the phrase “first vector” refers to the vector which comprises a PLE transposon; the phrase “second vector” refers to the vector which comprises a nucleotide sequence that encodes a PLE transposase. In one example, the transformation system may comprise b) a single vector comprising the PLE transposon as defined in the first aspect of the present disclosure and the nucleotide sequence that encodes the PLE transposase as defined in the second aspect. An exemplary transformation system comprising a single vector comprising a transposon and a transposase as defined herein is illustrated in FIG. 6.

In one example, the transformation system of the present disclosure may comprise a first vector may comprise SEQ ID NO: 4 (PLE-wu 5′ end) or SEQ ID NO: 24 (5′ end of truncated/short PLE-wu); or at least a sequence with at least 50%, at least 60%, at least 70%, or at least 80%, or at least 85%, or at least 90%, or at least 95%, or at least 97.5%, or at least 98%, or at least 99% identity to SEQ ID NO: 4 (PLE-wu 5′ end) or SEQ ID NO: 24 (5′ end of truncated/short PLE-wu); and/or SEQ ID NO: 5 (PLE-wu 3′ end) or SEQ ID NO: 25 (3′ end of truncated/short PLE-wu). In another example, the vector as described herein may comprise SEQ ID NO: 5 (PLE-wu 3′ end) or SEQ ID NO: 25 (3′ end of truncated/short PLE-wu); or at least a sequence with at least 50%, at least 60%, at least 70%, or at least 80%, or at least 85%, or at least 90%, or at least 95%, or at least 97.5%, or at least 98%, or at least 99% identity to SEQ ID NO: 5 (PLE-wu 3′ end) and/or SEQ ID NO: 4 (PLE-wu 5′ end); or SEQ ID NO: 24 (5′ end of truncated/short PLE-wu) and/or SEQ ID NO: 25 (3′ end of truncated/short PLE-wu). In another example, the transformation system of the present disclosure the second vector may comprise a nucleotide sequence which encodes an amino acid sequence of SEQ ID NO: 2; or SEQ ID NO: 27 or an amino acid sequence with at least 70%, or at least 80%, or at least 85%, or at least 90%, or at least 95%, or at least 97.5%, or at least 98%, or at least 99% identity to SEQ ID NO: 2; or SEQ ID NO: 27. In another example, the PLE transposase may comprise a nucleotide sequence of SEQ ID NO: 3 or SEQ ID NO: 28; or a sequence with at least 70%, or at least 80%, or at least 85%, or at least 90%, or at least 95%, or at least 97.5%, or at least 98%, or at least 99% identity to SEQ ID NO: 3; or SEQ ID NO: 28.

In one example, the transformation system may comprise a first vector according to the first aspect, wherein the first vector comprises a PLE transposon; and a nucleic acid encoding a transposase as defined in the second aspect or a transposase protein for delivery with the first vector. As used herein, the term “delivery” in the present disclosure refer to the action of transporting or delivering and may include any one of the following examples:

i. the first vector is to be delivered into a cell together with the nucleic acid encoding the transposase or the protein;

ii. the first vector is to be delivered into the cell before delivery of the nucleic acid encoding the transposase or the protein into the cell; and/or

iii. the first vector is to be delivered into the cell after delivery of the nucleic acid encoding the transposase or the protein into the cell.

The transformation system of the present disclosure may be used for various transformation purposes. For example, the transformation system of the present disclosure may be used in germline transformation, gene tagging, in gene or enhancer trapping, genomic manipulation, gene therapy, producing transgenic non-human animals, and/or producing induced pluripotent stem cells. When the transformation system is used for producing induced pluripotent stem cells, the stem cells may/may not comprise a transgene. Thus, the transformation system of the present disclosure may be used to create transgene-free induced pluripotent stem cells (IPS) cells.

To provide a convenient tool, the present disclosure, in another aspect, also provides a kit for transforming a cell comprising one or more vector as described herein. In one example, the kit may comprise a first vector as described in the first aspect, wherein the first vector may comprise a PLE transposon; and a second vector according to the second aspect, wherein the second vector may comprise a nucleotide sequence that encodes a PLE transposase. In one example, the kit of the present disclosure may comprise a first vector may comprise SEQ ID NO: 4 (PLE-wu 5′ end) or SEQ ID NO: 24 (5′ end of truncated/short PLE-wu); or at least a sequence with at least 50%, at least 60%, at least 70%, or at least 80%, or at least 85%, or at least 90%, or at least 95%, or at least 97.5%, or at least 98%, or at least 99% identity to SEQ ID NO: 4 (PLE-wu 5′ end) or SEQ ID NO: 24 (5′ end of truncated/short PLE-wu); and/or SEQ ID NO: 5 (PLE-wu 3′ end) or SEQ ID NO: 25 (3′ end of truncated/short PLE-wu). In another example, the vector as described herein may comprise SEQ ID NO: 5 (PLE-wu 3′ end) or SEQ ID NO: 25 (3′ end of truncated/short PLE-wu); or at least a sequence with at least 50%, at least 60%, at least 70%, or at least 80%, or at least 85%, or at least 90%, or at least 95%, or at least 97.5%, or at least 98%, or at least 99% identity to SEQ ID NO: 5 (PLE-wu 3′ end) and/or SEQ ID NO: 4 (PLE-wu 5′ end); or SEQ ID NO: 24 (5′ end of truncated/short PLE-wu) and/or SEQ ID NO: 25 (3′ end of truncated/short PLE-wu). In another example, the kit of the present disclosure may comprise a second vector may comprise a nucleotide sequence which encodes an amino acid sequence of SEQ ID NO: 2; or SEQ ID NO: 27 or an amino acid sequence with at least 70%, or at least 80%, or at least 85%, or at least 90%, or at least 95%, or at least 97.5%, or at least 98%, or at least 99% identity to SEQ ID NO: 2; or SEQ ID NO: 27. In another example, the PLE transposase may comprise a nucleotide sequence of SEQ ID NO: 3 or SEQ ID NO: 28; or a sequence with at least 70%, or at least 80%, or at least 85%, or at least 90%, or at least 95%, or at least 97.5%, or at least 98%, or at least 99% identity to SEQ ID NO: 3; or SEQ ID NO: 28.

In one aspect, there is provided a kit for transforming cell that may comprise a single vector comprising the PLE transposon as defined in the first aspect of the present disclosure and the nucleotide sequence that encodes the PLE transposase as defined in the second aspect.

In one example, the kits of the present disclosure may further comprise one or more primers such as SEQ ID NOs: 6 to 23. The kits may also further comprise reagents for performing transfections and/or polymerase chain reactions (PCR).

In another aspect, there is provided an oligonucleotide primer comprising the nucleotide sequence of any one of SEQ ID NOs: 6 to 23. As used herein, the term “primer” refers to an oligonucleotide that can be extended by a nucleic acid polymerase and would be easily recognized by a person skilled in the art to be useful in methods of amplifying a nucleotide sequence.

In another aspect, there is provided a method of amplifying a nucleotide sequence of SEQ ID NO: 1. The term “amplifying”, or its grammatical variants thereof, as used herein refers to a nucleic acid or nucleic acid reactions that occurs in in vitro methods of making copies of a particular nucleic acid, such as a target nucleic acid. Numerous methods of amplifying nucleic acids are known in the art, and amplification reactions include polymerase chain reactions (PCR), ligase chain reactions, strand displacement amplification reactions, rolling circle amplification reactions, transcription-mediated amplification methods, or loop mediated amplification methods. A “copy” does not necessarily mean perfect sequence complementarity or identity to the target sequence. For example, copies can include nucleotide analogs such as deoxyinosine or deoxyuridine, intentional sequence alterations (such as sequence alterations introduced through a primer comprising a sequence that is hybridizable, but not complementary, to the target sequence), and/or sequence errors that occur during amplification. In the present disclosure, the method of this aspect may comprise performing a polymerase chain reaction, using a forward primer comprising the nucleotide sequence of SEQ ID NO: 6 and a reverse primer comprising the nucleotide sequence of SEQ ID NO: 7.

In another aspect, there is provided a method of amplifying a nucleotide sequence of SEQ ID NO: 3. In the present disclosure, the method of this aspect may comprise performing a polymerase chain reaction using a forward primer comprising nucleotide sequence of SEQ ID NO: 8 and a reverse primer comprising nucleotide sequence of SEQ ID NO: 9.

In yet another aspect, there is provided a method of amplifying a nucleotide sequence of SEQ ID NO: 4. In the present disclosure, the method may comprise performing a polymerase, chain reaction using a forward primer comprising nucleotide sequence of SEQ ID NO: 10 and a reverse primer comprising nucleotide sequence of SEQ ID NO: 11 or SEQ ID NO: 20.

In yet another aspect, there is provided a method of amplifying a nucleotide sequence of SEQ ID NO: 5. In the present disclosure, the method may comprise performing a polymerase chain reaction using a forward primer comprising nucleotide sequence of SEQ ID NO: 12 and a reverse primer comprising nucleotide sequence of SEQ ID NO: 13.

In another aspect, there is provided a method of transforming a cell with a gene. In one example, the method may be an in vitro method. The method may comprise a) transfecting the cell with a first vector according to the first aspect, wherein the first vector comprises the 5′ and 3′ ends of a PLE transposon; and transfecting the cell with a second vector according to the second aspect, wherein the second vector comprises a nucleotide sequence that encodes a PLE transposase. In one example, the method may comprise transfecting the cell with a single vector, wherein the single vector comprises the 5′ and 3′ ends of the PLE transposon and the nucleotide sequence that encodes the PLE transposase.

In one example, the first vector may comprise SEQ ID NO: 4 (PLE-wu 5′ end) or SEQ ID NO: 24 (5′ end of truncated/short PLE-wu); or at least a sequence with at least 50%, at least 60%, at least 70%, or at least 80%, or at least 85%, or at least 90%, or at least 95%, or at least 97.5%, or at least 98%, or at least 99% identity to SEQ ID NO: 4 (PLE-wu 5′ end) or SEQ ID NO: 24 (5′ end of truncated/short PLE-wu); and/or SEQ ID NO: 5 (PLE-wu 3′ end) or SEQ ID NO: 25 (3′ end of truncated/short PLE-wu). In another example, the vector as described herein may comprise SEQ ID NO: 5 (PLE-wu 3′ end) or SEQ ID NO: 25 (3′ end of truncated/short PLE-wu); or at least a sequence with at least 50%, at least 60%, at least 70%, or at least 80%, or at least 85%, or at least 90%, or at least 95%, or at least 97.5%, or at least 98%, or at least 99% identity to SEQ ID NO: 5 (PLE-wu 3′ end) and/or SEQ ID NO: 4 (PLE-wu 5′ end); or SEQ ID NO: 24 (5′ end of truncated/short PLE-wu) and/or SEQ ID NO: 25 (3′ end of truncated/short PLE-wu). In another example, the second vector may comprise a nucleotide sequence which encodes an amino acid sequence of SEQ ID NO: 2; or SEQ ID NO: 27 or an amino acid sequence with at least 70%, or at least 80%, or at least 85%, or at least 90%, or at least 95%, or at least 97.5%, or at least 98%; or at least 99% identity to SEQ ID NO: 2; or SEQ ID NO: 27. In another example, the PLE transposase may comprise a nucleotide sequence of SEQ ID NO: 3 or SEQ ID NO: 28; or a sequence with at least 70%, or at least 80%, or at least 85%, or at least 90%, or at least 95%, or at least 97.5%, or at least 98%, or at least 99% identity to SEQ ID NO: 3; or SEQ ID NO: 28.

As mentioned previously, the vectors of the present disclosure uniquely exhibit traceless excision activity that restores the original TTAA target sequence upon excision. Thus, in one example, the gene of interest may be integrated at TTAA sites in the chromosomes of the cell.

In yet another aspect, there is provided a method of transforming a cell with a gene. The method may be performed in vitro. In this aspect, the method may comprise the steps of transfecting the cell with a first vector according to the first aspect, wherein the first vector comprises the 5′ and 3′ ends of a PLE transposon; and delivering a nucleic acid encoding a transposase as defined in the second aspect or a transposase protein either together with the first vector or before transfection of the first vector or after transfection of the first vector.

In yet another aspect, there is provided a method of transforming a cell with a gene. In one example, the method may be performed in vitro. In this aspect, the method may comprise the steps of delivering a nucleic acid encoding a transposase as defined in the first aspect or a transposase protein into the cell, wherein the cell already comprises the 5′ and 3′ ends of a PLE transposon.

The method of transforming a cell as described herein may produce a stably transfected cell which resulted from the stable integration of a gene of interest into the cell. As used herein, the term “stable integration” of a polynucleotide into a cell refers to the introduction of a polynucleotide into a chromosome or mini-chromosome of the cell and, therefore, becomes a relatively permanent part of the cellular genome. Although episomes, such as plasmids, can sometimes be maintained for many generations (particularly if kept under selective pressure), genetic material carried episomally is generally more susceptible to loss than chromosomally-integrated material. Also, the chromatin structure of eukaryotic chromosomes can influence the level of expression of an integrated polynucleotide. Such chromatin-induced effects can diminish or enhance the relative degree to which an integrated polynucleotide is expressed. Typically, a number of integrated clones are produced and clones exhibiting desirable levels of expression under production conditions are selected.

As would be readily understood by the person skilled in the art, the term “nucleic acid”, “nucleotide” or “polynucleotide” as used herein refers to polymeric form of nucleotides of any length, either ribonucleotides or deoxyribonucleotides, or analogs or derivatives thereof. The terms also includes, but not limited to, double- and single-stranded DNA, as well as double- and single-stranded RNA, and RNA-DNA hybrids. It also includes transcriptionally-activated polynucleotides such as methylated or capped polynucleotides. In one example, when used in the methods of the present invention, the term “nucleic acid” may be mRNA or DNA.

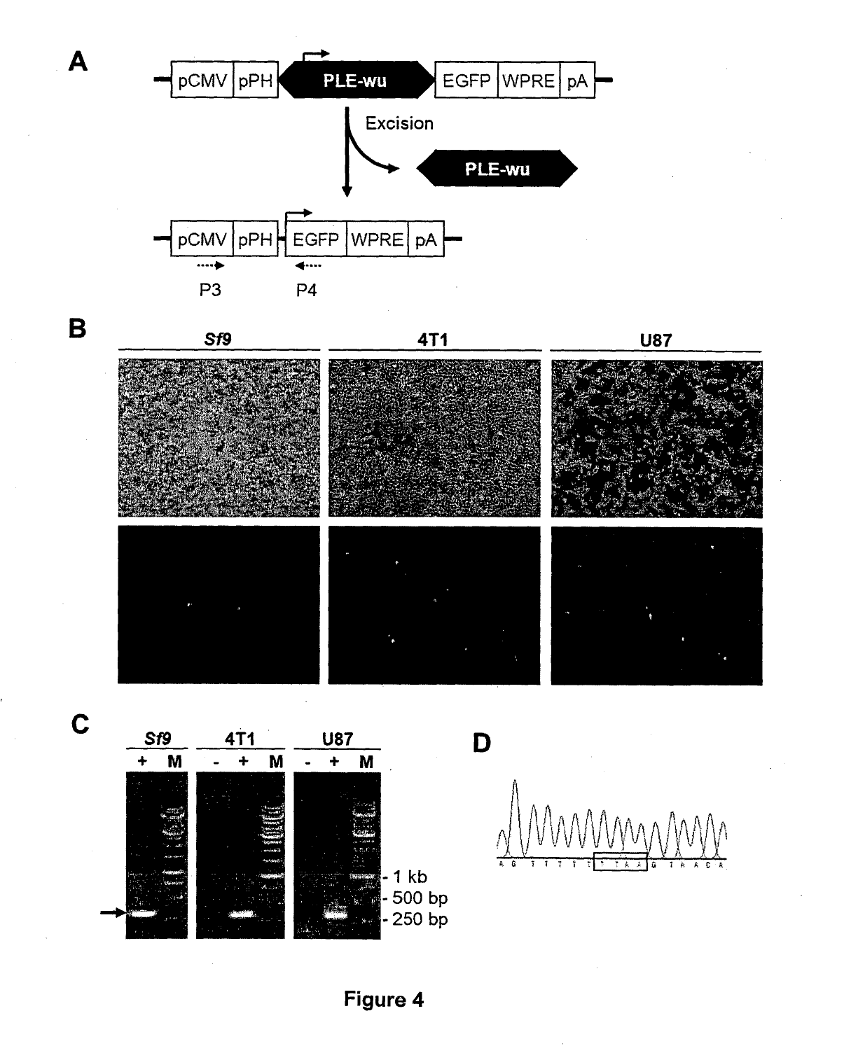

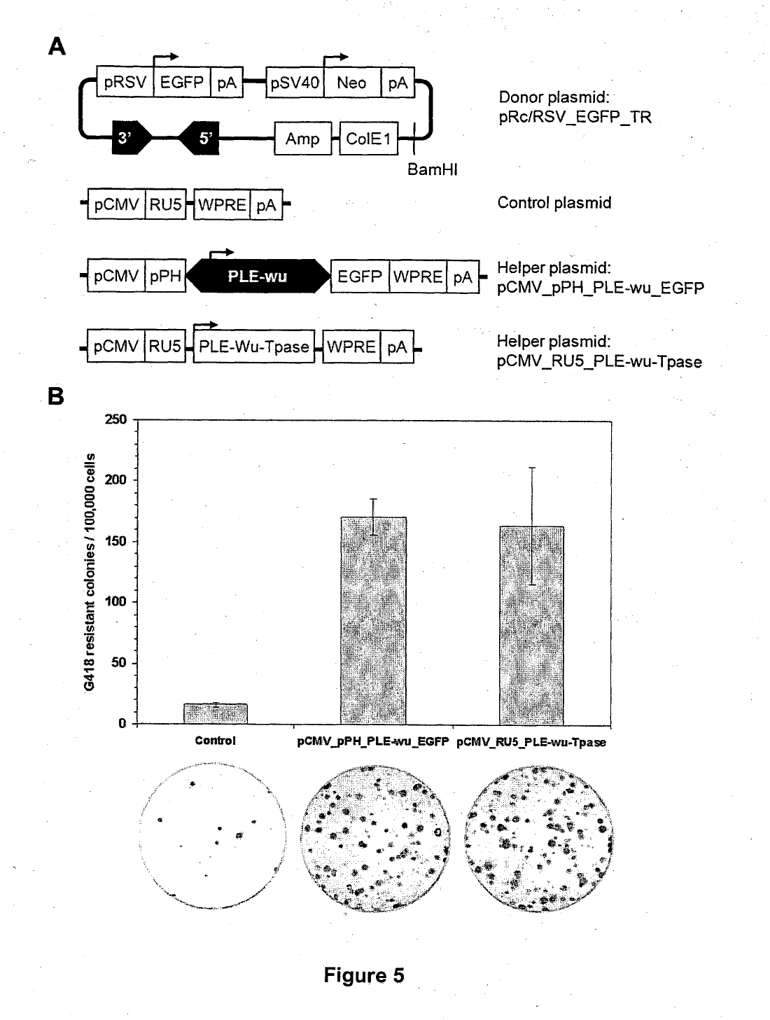

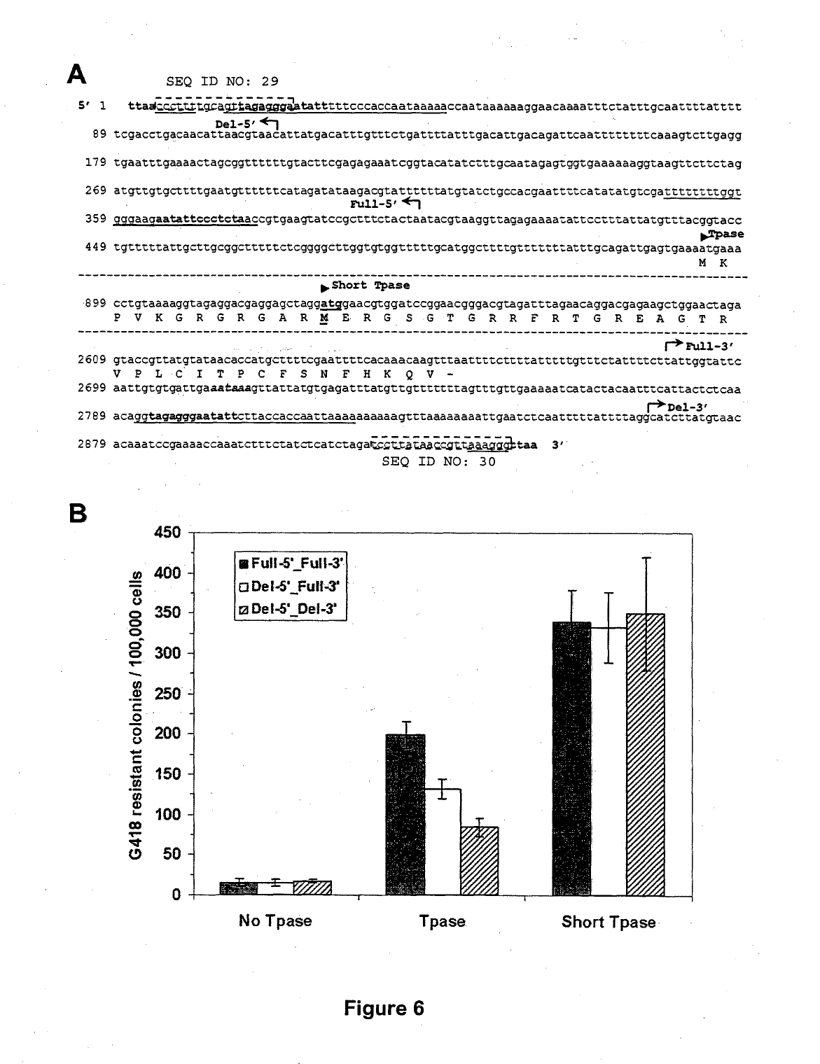

In one example, the method of the present invention may further comprise determining whether a gene of interest has been successfully excised from the chromosomes of the cell by a method commonly used in the art. In one example, as illustrated in the Example section of the present disclosure, the method may include performing a polymerase chain reaction with primers flanking the gene of interest; determining the size of the amplified polymerase chain reaction products obtained; and comparing the size of products obtained with a reference size, wherein if the size of the products obtained matches the expected size, then the gene of interest was successfully excised (see for example FIGS. 4 and 5). When determining whether a gene of interest has been successfully excised from the chromosome, primers used may include the nucleotide sequence of any one of SEQ ID NOs: 14 to 17.

In another example, the method of the present disclosure may further comprise determining where a gene of interest has been inserted into the chromosomes of the cells by a method commonly used in the art. As illustrated in the Example section of the present disclosure, the method may include performing a polymerase chain reaction with primers flanking the gene of interest and identifying the insertion site of transposition event. When determining the insertion site of a transposition event, primers used may include the nucleotide sequence of any one of SEQ ID NOs: 26 and 27.

When truncated transposons and/or transposase are used, the truncated transposons may be detected using primers that may include the nucleotide sequence of any one of SEQ ID NOs: 21 and 22. In another example, the nucleotide sequence which encodes the truncated transposase may be detected using primers that may include the nucleotide sequence of any one of SEQ ID NOs: 18 and 19.

In one example, there is provided a host cell comprising a vector as described herein. As used herein, the terms “host cells”, “cell lines”, “cell cultures”, and other such terms denote a prokaryotic or eukaryotic cell. In one example, the host cell may be a high eukaryotic cell. In another example, the cell may be mammalian cells, which can be used as recipients for recombinant vectors or other transfer polynucleotides, and include the progeny of the original cell that was transduced. It is understood that the progeny of a single cell are not necessarily completely identical in morphology or in genomic complement to the original parent cell. In one example, the host cell may comprise a transgene introduced by the vector as described herein. As used herein, the term “transgene” refers to a polynucleotide to be delivered to cells via a vector as described herein and can, comprise a coding sequence of interest in gene therapy. This may also be referred to as a “target polynucleotide” or a “therapeutic transgene”.

In yet another aspect, there is provided a non-human transgenic organism that may comprise cells which have been transformed by the methods of the present disclosure. In one example, the organism may be a mammal or an insect. When the organism is a mammal, the organism may include, but is not limited to, a mouse, a rat, a monkey, a dog, a rabbit and the like. When the organism is an insect, the organism may include, but is not limited to, a fruitfly, a mosquito, a bollworm and the like.

The invention illustratively described herein may suitably be practiced in the absence of any element or elements, limitation or limitations, not specifically disclosed herein. Thus; for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### EXPERIMENTAL SECTION

Materials and Methods

Cell Lines

Spodoptera frugiperda (Sf9) insect cells were cultured in spinner flasks using Sf-900 II SFM serum free medium (Invitrogen, Carlsbad, Calif.). U87 human glioma cells and 4T1 mouse mammary tumor cells were maintained in Dulbecco's modified Eagle's medium (DMEM) supplemented with 10% fetal bovine serum (FBS), penicillin (100 units/ml) and streptomycin (100 μg/ml).

PCR and Plasmid Construction

PLE-wu transposon was amplified from genomic DNA of baculovirus mutant dFP1 using primers GCGGAATTCGCAACAGAGCGTCGCGAG (SEQ ID NO: 6) and GCGGAATTCAGCGGAGCAAAAGCTGTTAC (SEQ ID NO: 7). The 708-residue transposase of PLE-wu was amplified using primers CGCGAATTCGCCACCATGAAAATACTTAGAGATGACG (SEQ ID NO: 8) and GCGGAATTCTTAAACTTGTTTGTGAAAATTCG (SEQ ID NO: 9). The 576-residue transposase was amplified using primers CGCGAATTCGCCACCATGGAACGTGGATCCGGAAC (SEQ ID NO: 18) and GCGGAATTCTTAAACTTGTTTGTGAAAATTCG (SEQ ID NO: 19). The amplifications were performed by initial denaturing of template DNA at 94° C. for 3 minutes, followed by 30 cycles of denaturing at 94° C. for 30 seconds, annealing at 55° C. for 30 seconds and extending at 72° C. for 3 minutes. The final extension was performed at 72° C. for 10 min. pBacPAK9 plasmid (Clontech, Mountain View, Calif.) was modified by inserting the cytomegalovirus promoter (pCMV) in front of the baculovirus polyhedrin promoter (pPH). Woodchuck hepatitis virus posttranscriptional regulatory element (WPRE) and SV40 polyadenylation (pA) signal were placed at the end of the multiple cloning site. pCMV_pPH_PLE-wu_EGFP was constructed by inserting the whole PLE-wu transposon and EGFP gene between pPH and WPRE of the resulting plasmid. pCMV_RU5_PLE-wu-Tpase was constructed by insert CMV promoter and R segment and part of the U5 sequence (RU5) of the HTLV Type 1 Long Terminal Repeat downstream of pPH, followed by the PLE-wu-Tpase, WPRE and pA in pBacPAK9.

pRc/RSV_EGFP_TR, the donor plasmid for transposition, was constructed by placing EGFP downstream of RSV promoter, the 5′ and 3′ end of PLE-wu (includes inverted terminal repeat, sub-terminal directed and inverted repeats) were placed upstream of RSV promoter. The 5′ and 3′ end of PLE-wu, separated by the 220-bp sequence upstream and downstream of transposon in fp25k gene of baculovirus, were amplified from dFP1 using PCR with primers GCTGAATTCAACACGCGACTCTTTTCGTC (SEQ ID NO: 10) and GCGACTAGTAGAAAGCGGATACTTCAC (SEQ ID NO: 11) or GCGACGCGTAGTAGAAAGCGGATACTTCAC (SEQ ID NO: 20) (for Full-5′ end) and GCGACGCGTATTGGTATTCAATTGTGTGATTG (SEQ ID NO: 12) and GCTGAATTCAAGAATGGCAAACCAAGTCG (SEQ ID NO: 13) (for Full-3′ end). Sequences with deletions at the 5′ and 3′ terminals were amplified using primers GCGACGCGTTACGTTAATGTTGTCAGGTC (SEQ ID NO: 21) (for Del-5′) and GCGACGCGTGCATCTTATGTAACACAAATCC (SEQ ID NO: 22) (for Del-3′). The PCR conditions were similar to those used to amplify PLE-wu, with a shorter extension time of 30 seconds.

The four primers used to detect excision activity of PLE-wu transposon have the following sequence: CGCCTCGAGGACGTCTGGCAAGAATCACA (SEQ ID NO: 14; P1), CGCAAGCTTCACGACAGCAGGCTGAATAA (SEQ ID NO: 15; P2), GAACCGTCAGATCCGCTAGT (SEQ ID NO: 16; P3), and GAACTTCAGGGTCAGCTTGC (SEQ ID NO: 17; P4). The PCR condition using P1 and P2 was identical to the condition used to amplify. PLE-wu. The condition using P3 and P4 is similar, except that the annealing temperature is 58° C. and the extension time is 30 seconds for 40 cycles.

Plaque Assay

Sf9 insect cells were co-transfected with pCMV_pPH_PLE-wu_EGFP and linearized baculovirus genome BacPAK6 according to the procedures suggested by the manufacture (Clontech). For plaque assay, Sf9 cells were seeded in 6-well plates at 50% confluence. One hour later the medium was replaced serial-diluted baculovirus in 1 ml of fresh sfm-900 II and incubated for one hour at room temperature. The cells were washed once with fresh sfm-900 II medium before adding 1% nutrient agarose overlay. Plaques were observed under a microscope 6 days later.

Colony Formation Assay

1×10⁵ of U87 or 4T1 cells were co-transfected with 0.4 μg of donor and 0.4 μg of helper plasmid using Lipofectamine 2000 (Invitrogen, Carlsbad, Calif.) in 24-well plates. Twenty-four hours later, the cells were trypsinized and half of the cells were transferred to 100 mm dishes in culture medium containing 0.3 mg/ml of G418. Two weeks later, drug-resistant colonies were fixed in 4% paraformaldehyde for 10 minutes and stained with 0.2% methylene blue for 1 hour.

Plasmid Rescue Assay

U87 cells were co-transfected with donor and helper plasmid. After 2 weeks selection in 0.3 mg/ml of G418, drug-resistant cells were harvested through trypsinization and their genomic DNAs were purified using tissue DNA extraction kit (Qiagen, Valencia, Calif.). Two μg genomic DNA were digested with 20 units of BamHI for 12 hours and purified with gel purification kit (Marligen Biosciences Inc, Ijamsville, Md.). The digested genomic DNA fragments were circularized with 20 units of T4 DNA ligase in 200 μl reaction volume at 4 degree for 48 hours and purified through the gel purification kit. One hundred ng of purified DNA were used to transform Top10 competent cells (Invitrogen, Carlsbad, Calif.). The emerged colony was cultured and its plasmid DNA was sequenced to identify the insertion site of transposition event. The 3′ end of PLE-wu and its nearby chromosome region was amplified using primer CACAAATCCGAAAACCAAATC (SEQ ID NO: 26) and TGAACCCACCACATTTCAGA (SEQ ID NO: 27).

The sequence of PLE-wu has been submitted to the DDBJ/EMBL/GenBank databases under accession number AB841319.

Results

Structure of the New Transposon

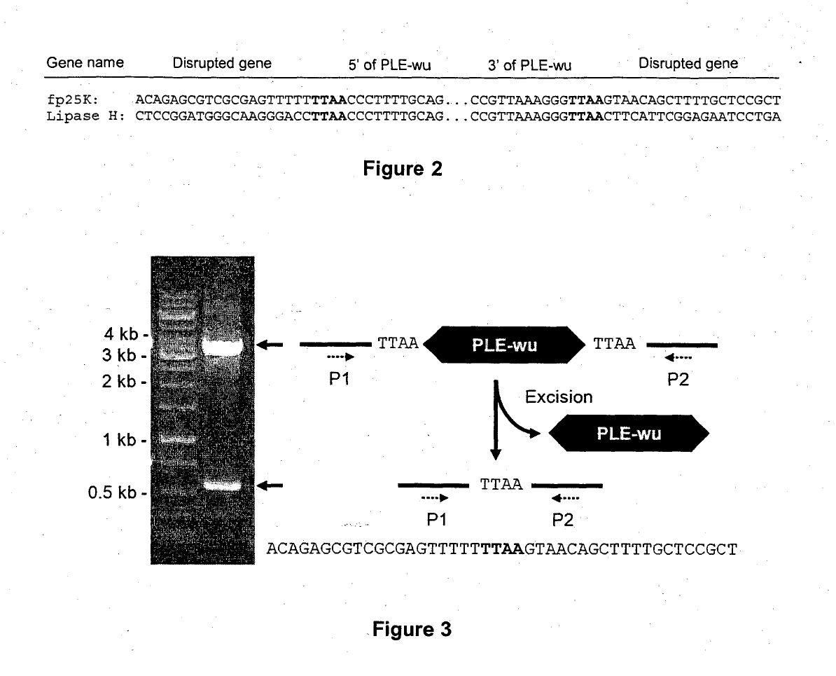

During direct genomic sequencing of an AcMNPV baculovirus clone dFP1, the inventors of the present disclosure found a 3-kb foreign sequence insert in the fp25k gene. The insertion results in duplication of TTAA at the junctions (FIGS. 1 and 2), a typical characteristic of piggyBac-like element transposition event. This new piggyBac-like element, termed PLE-wu, has a 6-bp inverted terminal repeat (CCCTTT/AAAGGG) next to the TTAA insertion site. A closer examination at the 5′ and 3′ end of PLE-wu revealed that it has a 32-bp imperfect sub-terminal direct repeat at both ends. Interestingly, this new transposon also has another 32-bp sequence downstream of 5′ sub-terminal direct repeat, which can form an imperfect inverted repeat with the sub-terminal direct repeats. All these sub-terminal repeats share a common 13-bp stretch of perfect match as the core sequence (FIG. 1).

PLE-wu contains a long open reading frame (ORF) predicted to encode a protein of 708 amino acids. BLAST results showed that it belongs to the DDE transposase superfamily. Its C-terminal, rich in cystine, formed a zinc-ribbon domain frequently presented in other transposases. The transposase of PLE-wu shows the highest similarity to IDT subfamily of PLEs, with about 56% identity at the DDE and zinc-ribbon domains. However, its similarity with piggyBac is low, which is only about 25% at the two domains. Compared to other transposase of PLEs, the N-terminal of the PLE-wu transposase is about 100 residues longer and showed no similarity to other proteins. Interestingly, an in-frame start codon at 396 bp downstream of the first start codon can potentially encode a short form of transposase (576 residues) similar in length to other PLE transposase (FIG. 1). At the 3′ end of PLE-wu, there is a polyadenylation signal (AATAAA) after the stop codon. This suggests that PLE-wu may be an active transposon.

Traceless Excision Active of PLE-Wu in Insect and Mammalian Cells

One unique feature of piggyBac transposon is its traceless excision, which restores the sequence at the insert site back to the original TTAA sequence after removal of the transposon. When PCR reactions were performed on the genomic DNA of dFP1 mutant virus using primers surrounding PLE-wu, two PCR products were obtained. One had the expected size of 3.5 kb, and the other one had a size of 0.6 kb, which is the size without PLE-wu (FIG. 3). The sequencing result of the small PCR product showed that it is the original sequence in fp25k (FIG. 3), probably due to the traceless excision of the PLE-wu.

To further confirm the traceless excision activity of PLE-wu in insect cells, pCMV_pPH_PLE-wu_EGFP plasmid was constructed. It has a backbone of pBacPAK9 transfer vector with a CMV and polyhedrin dual promoter for expression in both mammalian cells and insect cells (FIG. 4A). The EGFP expression is disrupted by the insertion of PLE-wu between the dual promoter and EGFP coding sequence. Upon excision of PLE-wu, the normal translation of EGFP will be restored, resulting in high expression level of EGFP due to the strong promoters (FIG. 4A). pCMV-pPH-PLE-wu_EGFP was co-transfected with the linearized viral genome to produce recombinant baculovirus. Plaque assays of the virus showed that the majority of infected Sf9 cells did not express EGFP, but a few cells did have high level expression of EGFP (FIG. 4B). Using the viral DNA as a template and primers that anneal to the CMV promoter and EGFP coding sequence, the inventors of the present disclosure detected a PCR product of 310 bp in length, the correct size after the excision of PLE-wu. Sequencing of this PCR product confirmed that it is the result of the traceless excision of PLE-wu, which restored the original TTAA insertion site sequence (FIGS. 4C and D).

To test the excision activity of PLE-wu in mammalian cells, U87 human glioma cells or 4T1 mouse mammary tumor cells were transfected with pCMV_pPH_PLE-wu_EGFP. A few green cells were observed on the second day after transfection (FIG. 4B). PCR amplification of the genomic DNAs produced a 310-bp product and the sequencing results revealed the traceless excision of PLE-wu (FIGS. 4C and D), demonstrating the traceless excision activity of PLE-wu in mammalian cells.

Insertion Activity of PLE-Wu in Mammalian Cells

After demonstrated the traceless excision activity of PLE-wu in mammalian cells, the possibility of whether PLE-wu also had the insertion activity in mammalian cells was also investigated. To test this, the two-plasmid donor-helper system was utilized. pRc/RSV_EGFP_TR, the donor plasmid, contains the expression cassettes for EGFP and neomycin resistant gene. The 400 bp at the 5′ end (Full-5′) and 250 bp at the 3′ end of PLE-wu (Full-3′), including the terminal repeat, sub-terminal repeats, and the polyadenylation signal were placed between the EGFP expression cassette and the ampicillin resistant gene. The 5′ and 3′ part of PLE-wu were placed in the inverted orientation and separated by the 220 bp sequence in fp25k surrounding PLE-wu in the baculovirus mutant (FIGS. 5A and 6A). Two types of helper plasmid, pCMV_pPH_PLE-wu_EGFP and pCMV_RU5_PLE-wu-Tpase, were tested. The latter has the 708-residue transposase (PLE-wu-Tpase) directly expressed from the CMV_RU5 promoter without the 5′ or 3′ terminal structures of transposon presented in pCMV_pPH_PLE-wu_EGFP (FIG. 5A). U87 human glioma cells were co-transfected with the donor and helper plasmids. The numbers of emerged G418 resistant colonies using pCMV_pPH_PLE-wu_EGFP and pCMV_RU5_PLE-wu-Tpase as helper plasmids were quite similar; both were about 10 times more than that of control plasmid without PLE-wu expression (FIG. 5B). Moreover, the majority of the drug resistant colonies were also EGFP positive, while only about half of the colonies showed green fluorescence in the control group (data not shown), suggesting the excision at the 5′ and 3′ end of PLE-wu and insertion of the entire plasmid including both the EGFP and Neo expression cassette in the presence of PLE-wu-Tpase.

To check the insertion site of the PLE-wu mediated transposition event in mammalian cells, plasmid rescue assay was performed. The genomic DNA of those G418 resistant cells was isolated, digested with BamHI and circularized to transform competent E. coli cells. One colony was obtained and the sequencing result showed that the 5′ end of insertion site is at lipase H gene on chromosome 3q27. When the 3′ end of the insertion site was amplified and sequenced, the duplication of TTAA insertion sequence of PLE-wu was observed (FIG. 2). These data demonstrate that transposase of F′LE-wu still retains insertion activity in mammalian cells.

To map the terminal sequences required for efficient transposition, donor plasmids with deletion at 5′ and 3′ end of PLE-wu was used in the two-plasmid donor-helper system (FIG. 6A). Compared to the construct with full length of 5′ and 3′ end terminals, transposition activity of the 708-residue transpose decreased to 70% when the 110 bp sequence at 5′ end paired with the full 3′ end terminal. Its transposition activity further decreased to 40% when the 3′ end was also reduced to 80 bp. Surprisingly, the short 576-residue transposase, translated from the downstream in-frame initiation codon, also had transposition activity in mammalian cells. Its activity was about 80% higher than that of 708-residue transpose and was not affected by the 5′ and 3′ terminal deletions tested here (FIG. 6B). These show that the two forms of transposase require different terminal sequences for efficient transposition.

Through direct genomic DNA sequencing of a mutant baculovirus, the inventors of the present disclosure discovered PLE-wu as a new class II transposon. PLE-wu was flanked by the TTAA target sequence in fp25K gene of mutant virus. Its terminal structure is more complex than piggyBac, containing not only the inverted terminal repeat, but also the inverted and direct sub-terminal repeats. The 6-bp inverted terminal repeat, shorter than the 13-bp one in piggyBac, also starts with CCCT as in piggyBac. For sub-terminal repeats, there are no obvious similarities to piggyBac or other PLEs.

PLE-wu contains a long ORF, followed by a TTATTT polyadenylation signal at the 3′ end. Sequence analysis shows that this transposase belongs to the family of piggyBac-like element, with high similarity to other PLEs at the DDE domain and the cystine-rich domain at C-terminal. Compared to other PLEs which have about 500 to 600 amino acids residues, the putative 708 residues-long transposase of PLE-wu is the largest among them. As deletions in the 5′ and 3′ end of terminal decreased its transposition activity, the extra long N-terminal portion, with no similar to other proteins, may be involved in binding to those complex terminal repeats. The finding that the 576-residue transpose has higher transposition activity is unexpected. Since most of mutations in the genome are deleterious to its host, the 708-residue transposase may evolve from the 576-residue one. With its reduced activity and requirement for extra elements in the terminals, the harmful effect from high frequency transposition will be reduced.

The transposase of PLE-wu is active in both insect and mammalian cells. Its excision activity resembles “traceless excision”, the unique feature of piggyBac. In mammalian cells, the transposase of PLE-wu can also carry out the second step of transposition, inserting the DNA in to a new location in the genome. The plasmid to chromosome transposition rate of PLE-wu is similar to that reported for piggyBac, which is about 10-fold higher than random integration. Apparently piggyBac requires no host factors for its transposition activity, as it is active in many species range from yeast to humans, and the purified transposase is active in vitro. It is very likely that the transposition activity of PLE-wu is also host factor-independent. Through plasmid rescue, the duplication of TTAA target site upon insertion was observed. piggyBac has a preference of insertion into regions near the transcription starting sites. The rescued clone obtained had the insertion site at lipase H gene on chromosome 3. In one example, PLE-wu may have insertion site preference in the genome.

It is very likely that the transposition activity of PLE-wu is also host factor-independent. The transposition rates of transposon in mammalian cells are quite low. Among Tol2, Sleeping Beauty and piggyBac, the three commonly used transposon system, piggyBac shows the highest activity. To improve the transposition rate, various point mutations have been introduced into the original transposase. The identification of this new active PLE-wu transposon, especially with its low sequence similarity with piggyBac, is useful in the design of new PLEs with higher activity, as well as the study of the mechanisms of PLE.

1. A vector, comprising a piggyBac like element (PLE) transposon.

2. The vector according to claim 1, wherein the 5′ and 3′ ends of the transposon are used to flank a gene which is to be transformed into a cell.

3. The vector according to claim 1, wherein the PLE transposon is not a piggyBac transposon and/or wherein the PLE transposon does not comprise sub-terminal repeats.

4. The vector according to claim 1, wherein the PLE transposon comprises at least one pair of an inverted terminal repeat at the 5′ and 3′ ends of the transposon.

5. The vector according to claim 4, wherein the inverted terminal repeat is an imperfect inverted terminal repeat.

6. The vector according to claim 5, wherein the imperfect inverted terminal repeat comprises cccttttgcagttagaggga (SEQ ID NO: 28) and tccttataaccgttaaaggg (SEQ ID NO: 29).

7. (canceled)

8. The vector according to claim 1, wherein the PLE transposon comprises SEQ ID NO:1; or a sequence with at least 70%, at least 75%, at least 80%, or at least 85%, or at least 90%, or at least 95%, or at least 98%, or at least 99% identity to SEQ ID NO: 1.

9. The vector, according to claim 1, comprising SEQ ID NO: 4 (PLE-wu 5′ end) or SEQ ID NO: 24 (5′ end of truncated/short PLE-wu); or a sequence with at least 70%, at least 75%, at least 80% or at least 85%, or at least 90%, or at least 95% identity to SEQ ID NO: 4; and/or SEQ ID NO: 5; SEQ ID NO: 24 (5′ end of truncated/short PLE-wu) and/or SEQ ID NO: 25 (3′ end of truncated/short PLE-wu); or a sequence with at least 80% or at least 85%, or at least 90%, or at least 95%, or at least 98%, or at least 99% identity to SEQ ID NO: 5 (PLE-wu 3′ end) or SEQ ID NO: 25 (3′ end of truncated/short PLE-wu).

10. A vector, comprising a nucleotide sequence encoding a piggyBac like element (PLE) transposase.

11. The vector according to claim 10, wherein the transposase is used to excise a gene flanked by the 5′ and 3′ ends of a PLE transposon and to integrate the gene into the chromosomes of a cell.

12. The vector according to claim 10, wherein the transposase is not a piggyBac transposase.

13. The vector according to claim 10, wherein the PLE transposase comprises an amino acid sequence of SEQ ID NO: 2 or SEQ ID NO: 27; or an amino acid sequence with at least 70%, at least 75%, at least 80%, or at least 85%, or at least 90%, or at least 95% identity, or at least 98%, or at least 99% to SEQ ID NO: 2 or SEQ ID NO: 27.

14. The vector according to claim 10, wherein the nucleotide sequence encoding the PLE transposase comprises SEQ ID NO: 3 or SEQ ID NO: 28 or a sequence with at least 70%, at least 75%, at least 80%, or at least 85%, or at least 90%, or at least 95% identity, or at least 98%, or at least 99% to SEQ ID NO: 3 or SEQ ID NO: 28.

15. (canceled)

16. The vector according to claim 11, wherein the PLE transposase includes a 1 to 350 residues, 50 to 150 residues or 132 residues deletion of the N-terminal of the PLE transposase, which enhances the activity of the PLE transposase.

17. The vector according to claim 16, wherein the PLE transposase comprises SEQ ID NO: 27.

18. The vector according to claim 17, wherein the PLE transposase comprises an amino acid sequence of SEQ ID NO: 25 or an amino acid sequence with at least 70%, at least 75%, at least 80%, or at least 85%, or at least 90%, or at least 95% identity, or at least 98%, or at least 99% to SEQ ID NO: 25.

19. The vector according to claim 1, to be used in combination with a vector, comprising a nucleotide sequence encoding a piggyBac like element (PLE) transposase.

20-27. (canceled)

28. A transformation system, comprising
a) a first vector, comprising a piggyBac like element (PLE) transposon; and
a second vector, comprising a nucleotide sequence encoding a piggyBac like element (PLE) transposase; or
b) a single vector comprising the piggyBac like element transposon and the nucleotide sequence that encodes the piggyBac like element transposase.

29. The transformation system according to claim 28, wherein the first vector comprises any one of SEQ ID NOs: 4 and 5; or a sequence with at least 70%, at least 75%, at least 80% or at least 85%, or at least 90%, or at least 95% identity, or at least 98%, or at least 99% to any one of SEQ ID NOs: 4 and 5.

30. The transformation system according to claim 28, wherein the second vector comprises either SEQ ID NO: 3; or a sequence with at least 70%, at least 75%, at least 80% or at least 85%, or at least 90%, or at least 95%, or at least 98%, or at least 99% identity to SEQ ID NO: 3; or a nucleotide sequence which encodes an amino acid sequence of SEQ ID NO: 2; or a nucleotide sequence which encodes an amino acid sequence with at least 70%, at least 75%, at least 80% or at least 85%, or at least 90%, or at least 95%, or at least 98%, or at least 99% identity to SEQ ID NO: 2.

31-40. (canceled)

41. A kit for transforming a cell, comprising one or more vectors, wherein each vector comprises piggyBac like element (PLE) transposon.

42. The kit according to claim 41, comprising a first vector, comprising a piggyBac like element (PLE) transposon; and
a second vector, comprising a nucleotide sequence encoding a piggyBac like element (PLE) transposase.

43. The kit according to claim 42, wherein the first vector comprises any one of SEQ ID NOs: 4 and 5; or a sequence with at least 70%, at least 75%, at least 80% or at least 85%, or at least 90%, or at least 95% identity to any one of SEQ ID NOs: 4 and 5; optionally wherein the second vector comprises either SEQ ID NO: 3; or a sequence with at least 70%, at least 75%, at least 80% or at least 85%, or at least 90%, or at least 95%, or at least 98%, or at least 99% identity to SEQ ID NO: 3; or a nucleotide sequence which encodes an amino acid sequence of SEQ ID NO: 2; or a nucleotide sequence which encodes an amino acid sequence with at least 70%, at least 75%, at least 80% or at least 85%, or at least 90%, or at least 95%, or at least 98%, or at least 99% identity to SEQ ID NO: 2.

44-46. (canceled)

47. A kit for transforming a cell, comprising a single vector comprising a piggyBac like element transposon and a nucleotide sequence that encodes the piggyBac like element transposase.

48. (canceled)

49. A method of amplifying a nucleotide sequence of any one of SEQ ID NOs: 1, 3, 4 or 5, wherein the amplification of SEQ ID NO: 1 comprises performing a polymerase chain reaction using a forward primer comprising the nucleotide sequence of SEQ ID NO: 6 and a reverse primer comprising the nucleotide sequence of SEQ ID NO: 7; wherein the amplification of SEQ ID NO: 3 comprises performing a polymerase chain reaction using a forward primer comprising nucleotide sequence of SEQ ID NO: 8 and a reverse primer comprising nucleotide sequence of SEQ ID NO: 9; wherein the amplification of SEQ ID NO: 4 comprises performing a polymerase chain reaction using a forward primer comprising nucleotide sequence of SEQ ID NO: 10 and a reverse primer comprising nucleotide sequence of SEQ ID NO: 11 or SEQ ID NO: 20; wherein the amplification of SEQ ID NO: 5 comprises performing a polymerase chain reaction using a forward primer comprising nucleotide sequence of SEQ ID NO: 12 and a reverse primer comprising nucleotide sequence of SEQ ID NO: 13.

50-52. (canceled)

53. A method of transforming a cell with a gene, comprising:
a) transfecting the cell with a first vector, comprising a piggyBac like element (PLE) transposon, wherein the first vector comprises the 5′ and 3′ ends of a piggyBac like element (PLE) transposon; and
transfecting the cell with a second vector, comprising a nucleotide sequence encoding a piggyBac like element (PLE) transposase, wherein the second vector comprises a nucleotide sequence that encodes a piggyBac like element transposase; or
b) transfecting the cell with a single vector, wherein the single vector comprises the 5′ and 3′ ends of the piggyBac like element transposon and the nucleotide sequence that encodes the PLE transposase.

54. The method according to claim 53, wherein the first vector comprises any one of SEQ ID NOs: 4 and 5; or a sequence with at least 70%, at least 75%, at least 80% or at least 85%, or at least 90%, or at least 95%, or at least 98%, or at least 99% identity to any one of SEQ ID NOs: 4 and 5; optionally wherein the second vector comprises SEQ ID NO: 3; or a sequence with at least 70%, at least 75%, at least 80% or at least 85%, or at least 90%, or at least 95% or at least 98%, or at least 99% identity to SEQ ID NO: 3; or a nucleotide sequence which encodes an amino add sequence of SEQ ID NO: 2; or a nucleotide sequence which encodes an amino acid sequence with at least 70%, or at least 75%, at least 80%, or at least 85%, or at least 90%, or at least 95%, or at least 98%, or at least 99% identity to SEQ ID NO: 2.

55. (canceled)

56. (canceled)

57. A method of transforming a cell with a gene, comprising:
transfecting the cell with a first vector, wherein the first vector comprises the 5′ and 3′ ends of a piggyBac like element (PLE) transposon; and
delivering a nucleic acid encoding a nucleotide sequence encoding a piggyBac like element (PLE) transposase or a transposase protein either together with the first vector or before transfection of the first vector or after transfection of the first vector.

58. A method of transforming a cell with a gene, comprising:
delivering a nucleic acid encoding a nucleotide sequence encoding a piggyBac like element (PLE) transposase or a transposase protein into the cell, wherein the cell already comprises the 5′ and 3′ ends of a piggyBac like element (PLE) transposon.

59-61. (canceled)

62. A non-human transgenic organism comprising cells which have been transformed by a method of transforming a cell with a gene, comprising:
a) transfecting the cell with a first vector, comprising a piggyBac like element (PLE) transposon, wherein the first vector comprises the 5′ and 3′ ends of a piggyBac like element (PLE) transposon; and transfecting the cell with a second vector, comprising a nucleotide sequence encoding a piggyBac like element (PLE) transposase, wherein the second vector comprises a nucleotide sequence that encodes a piggyBac like element transposase; or
b) transfecting the cell with a single vector, wherein the single vector comprises the 5′ and 3′ ends of the of piggyBac like element transposon and the nucleotide sequence that encodes the PLE transposase.

63-65. (canceled)
